(19) United States
(12) Patent Application Publication
(10) Pub. No.: US 2026/0155246 A1
(43) Pub. Date: Jun. 4, 2026

(54) CUSTOMIZABLE CONTINUOUSLY UPDATED EVENT MONITORING FOR SCREENS ON COMPUTING DEVICE

(71) Applicant: Roche Diabetes Care, Inc., Indianapolis, IN (US)

(72) Inventors: Mark Gilmore Mears, Indianapolis, IN (US); Paul Steele, Carmel, IN (US); Ryan McKinney, Indianapolis, IN (US); Derek Adams, Carmel, IN (US)

(73) Assignee: Roche Diabetes Care, Inc., Indianapolis, IN (US)

(21) Appl. No.: 19/401,019

(22) Filed: Nov. 25, 2025

Related U.S. Application Data
(63) Continuation of application No. PCT/US2024/031656, filed on May 30, 2024.
Application No. PCT/US2024/031656 is a continuation of application No. PCT/US2023/024078, filed on May 31, 2023.

(51) Int. Cl. G16H 40/63

(52) U.S. Cl. CPC G16H 40/63

(57) ABSTRACT

A computing device may provide access to information related to the medical condition or health of the user in the form of an overlay notification when the computing device is displaying a graphical user interface. The overlay notification may be dynamically and/or continuously updated on the graphical user interface of the device in response to updated data. The overlay notification may be enabled/disabled in response to overlay notification triggering events/overlay notification removal events. When multiple types of information are to be displayed at a time, the computing device may group the information into a common overlay notification that may be updated dynamically and/or continuously in response to updated data. As the information that is provided in the overlay notification may be sensitive information, the information in the overlay notification may be prevented from being displayed until additional input is received from the user.

### CROSS-REFERENCE TO RELATED APPLICATIONS

This application is a continuation, under 35 U.S.C. § 111(a), of International Patent Application No. PCT/US24/31656, filed May 30, 2024, published as WO 2024/249623 on Dec. 5, 2024, and which claims priority to International Patent Application No. PCT/US23/24078, filed May 31, 2023, each of which is incorporated herein by reference in its entirety.

### BACKGROUND

People with medical conditions or other health-related conditions often need to monitor events to maintain their health. For example, a person with diabetes often needs to monitor their current glucose level regularly to make sure their glucose level does not go too high or too low. These people may perform continuous monitoring to determine whether applied therapeutic interventions are having the intended effect, or if additional interventions are needed to help treat their condition.

Individuals with medical conditions may use a number of devices to help them monitor their current condition and/or events that may occur over the course of a day. Individuals often use their mobile devices to monitor their current condition. In one example, a person with diabetes may use their mobile device (e.g., in combination with other sensor devices) to monitor their current blood glucose levels, insulin dosing information, information related to carbohydrate intake, and/or other information that may be helpful in treatment of the diabetic condition.

In order to view information related to a current condition on a mobile device, the user may need to unlock their mobile device and open the particular application to view certain information related to their condition. This may cause a delay in certain forms of treatment to assist the user in treating their condition. The application may also provide push notifications to the user to inform the user as to certain information relevant to treatment of their medical condition. These forms of notifications may cause the user to receive separate notifications related to the same condition each time there is a change in the user's condition or information related to the condition.

Preset notifications used to carry out health and wellness related activities (e.g., diagnostic, medication dose, diet, exercise) are common features used to enhance the user's treatment of their medical condition. However, in many cases and for a number of reasons, they are redundant, ignored and/or inconvenient to the user. Their inherent rigidity can result in the delay of desired health outcomes. Additionally, these notifications may cause sensitive health-related information of the user to be displayed such that this sensitive information may be viewed by others.

### SUMMARY

Embodiments are described herein for providing information to a user for assisting in treatment of a medical condition. The user may implement one or more computing devices for enabling them to properly treat their medical condition. For example, the user may implement their mobile device for monitoring information related to their medical condition or other health-related information.

The user's mobile device may provide an overlay notification related to the user's medical condition or other health-related information. In some embodiments, the user's mobile device may overlay the overlay notification on another graphical user interface being displayed on the mobile device, such as when the mobile device is displaying a lock screen in a locked state, a home screen, a call screen, or other graphical user interface. To enable access to the information related to their medical condition or other health-related information, the user may receive overlay notifications as push notifications and/or the overlay notifications may be updated based on updates to the information being provided.

An overlay notification may be dynamically or statically displayed on a graphical user interface provided on the mobile device. The graphical user interface over which the overlay notification is being displayed may be provided by the operating system or another application executing on the mobile device. The overlay notification may be generated and/or displayed by an application (e.g., diabetes-related application, exercise-related application, nutrition or mealtime-related application, another health-related application, or other type of diabetes application) residing locally on the mobile device and/or executing in the background of the mobile device. The overlay notification may be dynamically and/or continuously updated in response to updated information received by the mobile device. The overlay notification may be overlaid on a series of screens provided by one or more applications. The overlay notification may include, for example, a lock screen notification, a home screen notification, and/or a call screen notification.

A lock screen notification may be dynamically displayed on the lock screen of the mobile device. The lock screen notification may be generated and/or displayed by an application (e.g., diabetes-related application, exercise-related application, nutrition or mealtime-related application, another health-related application, or other type of diabetes application) residing locally on the mobile device and/or via information from one or more remote computing devices. The lock screen notification may be dynamically and/or continuously updated in response to updated information received by the mobile device. In one example, the mobile device may display the lock screen notification as a Continuously Updated Event Monitoring Entity (CUE-ME) notification. The CUE-ME notification may be dynamically updated based on data being monitored at the mobile device and/or one or more other devices. The user may monitor changes and/or progress of health-related events and/or other events from the lock screen of the mobile device.

The mobile device, or another computing device, may receive data related to a medical condition (e.g., a diabetic condition) of the user and/or other health related information for detecting triggering events for displaying the overlay notification. In response to detecting the overlay notification triggering event, the mobile device may dynamically display an overlay notification on the lock screen of the computing device. The overlay notification may be associated with a particular application (e.g., diabetes-related application). The overlay notification may be displayed for a period of time or until an overlay notification removal event is detected. For example, an overlay notification removal event may be detected based on the data that is received at the mobile device related to the medical condition of the user. The overlay notification may be continuously and/or dynamically updated in response to the received data related to the medical condition (e.g., a diabetic condition) of the user and/or other health related information while being displayed. The overlay notification may then, in response to detecting the overlay notification removal event, be removed from the lock screen of the mobile device. Though examples are provided for enabling/disabling the overlay notification based on data related to the medical condition of the user and/or other health related information, the overlay notification may be enabled/disabled based on an actuation from the user on the mobile device.

The information that is monitored for enabling the overlay notifications may include information related to a diabetic condition of the user. For example, the overlay notification triggering event may be detected when the received data from a continuous glucose monitor, or another monitoring device, indicates a glucose level of the user is above an upper threshold or below a lower threshold. The overlay notification triggering event may be detected based on one of an indication of an extreme diabetic state, an indication of an abnormal increase or decrease of blood glucose level within a predefined period of time, an indication of a performance of exercise by the person, an indication of mealtime, or an indication that the person received a predefined amount of short-acting insulin within a predefined period of time.

The information that is monitored for disabling the overlay notifications may also include information related to a diabetic condition of the user. For example, an overlay notification removal event may be detected based on one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.

When multiple types of information are to be displayed at a time, the mobile device, or other computing device, may group the information into a common overlay notification. Each type of information in the common overlay notification may be dynamically and/or continuously updated in the common overlay notification while being provided in the overlay notification. The mobile device may determine different types of information to be displayed that are each related to different overlay notifications and/or triggering events. The mobile device may determine that the different types of information are related and/or associated in memory for being displayed in a common overlay notification and dynamically display the information in the common diabetic overlay notification.

As the information that is provided in the lock screen notification may be sensitive medical and/or health-related information, or other information personal to the user, the information in the lock screen notification may be prevented from being displayed until additional input is received from the user. The mobile device may provide a notification that data related to the medical condition or the person is prepared to be displayed on the lock screen of the mobile device. The information may be prevented from being displayed in a readable format until the input is received from the user to enable the display of the information. The input from the user may authenticate the user to enable the information to be displayed in the lock screen notification. The lock screen notification may include an obscured display of the information on the lock screen, such as a blurred or redacted version of the information, for example.

### BRIEF DESCRIPTION OF THE DRAWINGS

FIGS. 1A-1C include perspective views of representative user environments.

FIG. 2A shows a flowchart of an example process for displaying, updating, and/or removing overlay notifications.

FIG. 2B shows a flowchart of another example process for displaying, updating, and/or removing overlay notifications.

FIG. 3 shows a flowchart of an example process for grouping information in diabetic overlay notifications.

FIG. 4 shows a flowchart of an example process for protecting user data when displaying on lock screen notifications.

FIGS. 5A-5F depict example GUIs illustrating overlay notifications provided on a display of a computing device.

FIG. 6 is a block diagram of an example computing device.

FIG. 7 is a block diagram of an example blood glucose monitoring device.

FIG. 8 is a block diagram of an example blood glucose meter (BGM) device.

### DETAILED DESCRIPTION

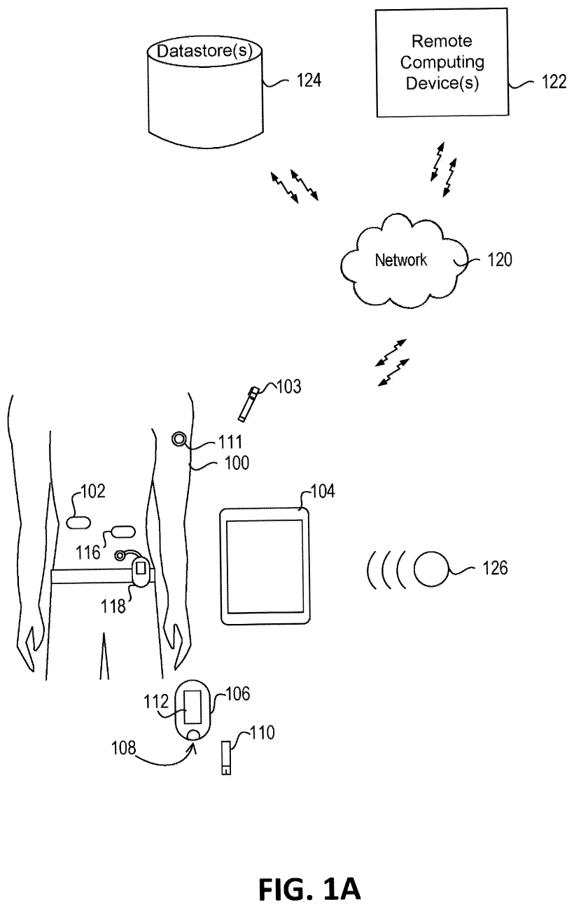

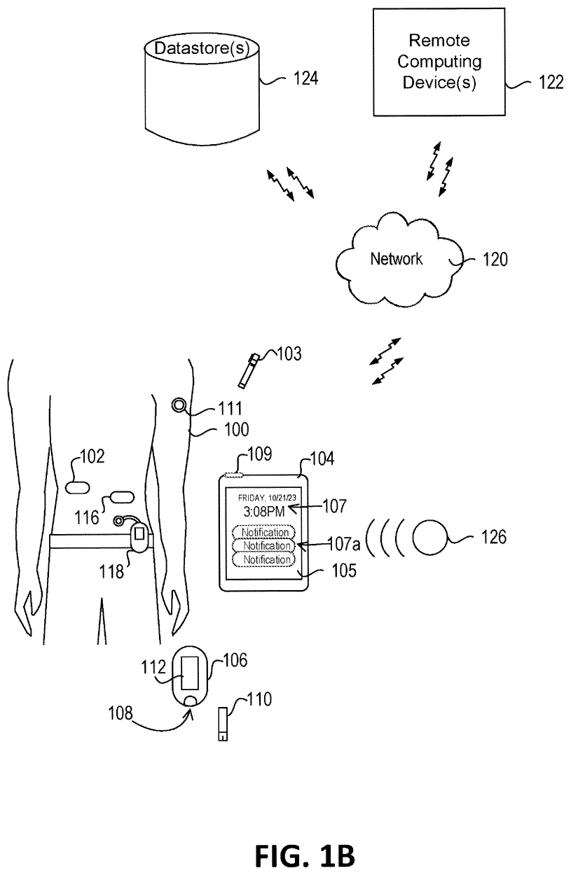

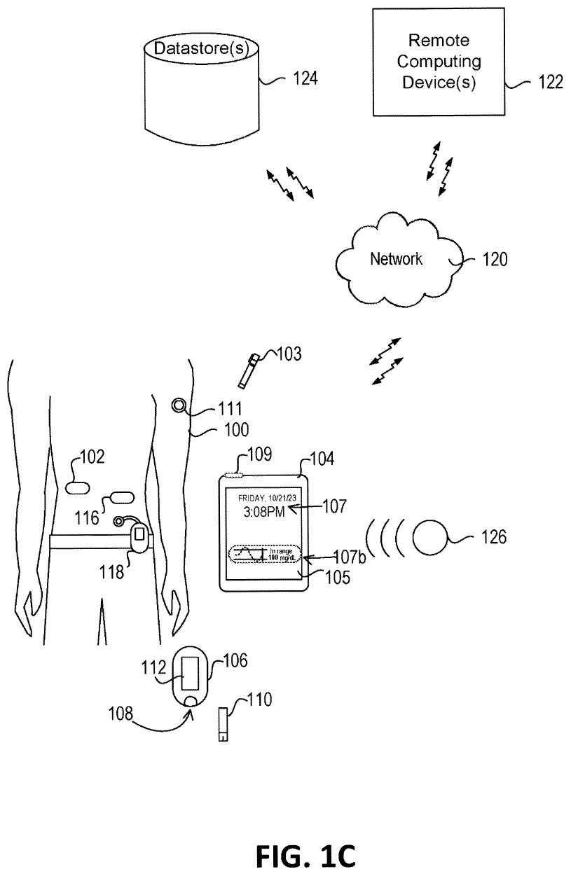

FIG. 1A is a perspective view of a representative user environment. As shown in FIG. 1A, a user 100 may be a person with diabetes or another medical condition. The person 100 with diabetes may use one or more devices to help monitor and/or treat the diabetic condition. The diabetic condition may include a metabolic syndrome, pre-diabetes, type 1 diabetes, type 2 diabetes, and/or gestational diabetes. The user 100 may be in an extreme diabetic state, such as hypoglycemia or hyperglycemia, when the blood glucose level of the user 100 is above or below a threshold blood glucose level. The user 100 may use a blood glucose monitoring device to monitor blood glucose levels.

A blood glucose monitoring device may include any device that detects and reports a level of glucose in the blood of the user, either through direct measurement of the blood or through an indirect detection process. A blood glucose level is also referred to as a blood sugar level. Examples of blood glucose monitoring devices include, but are not strictly limited to, continuous glucose monitoring devices, flash glucose monitoring devices, and blood glucose meters that provide a single measurement of blood glucose levels from a blood sample in a “spot” monitoring process. A blood glucose treatment device may include any device that treats a diabetic condition of the user, either through direct treatment into the blood or through an indirect treatment process or by providing information to allow for treatment.

In some embodiments, the blood glucose monitoring device and/or blood glucose treatment device is a pen device 103. The pen device 103 may include any injector pen capable of monitoring and/or managing insulin delivery. Example pen devices may communicate with an external device, such as the mobile device 104 and/or a remote computing device 122, to determine insulin levels, calculate doses, track doses, deliver insulin, and/or provide other information such as notifications or alerts. In some examples, pen devices may periodically communicate data indicating the blood glucose levels of the user 100 to an external device, such as a mobile device 104, for computing or storing the blood glucose levels of the user 100.

In some embodiments, the blood glucose monitoring device is a continuous glucose monitor (CGM) 102. The CGM 102 includes a subcutaneous sensor that is used to sense and monitor the amount of glucose in interstitial fluid of the user 100. The CGM 102 includes a transmitting device that is located directly over the sensor that wirelessly powers the data transfer from the sensor. The CGM 102 periodically communicates data indicating the blood glucose levels of the user 100 to an external device, such as a mobile device 104, for computing or storing the blood glucose levels of the user 100.

Some embodiments of the mobile device 104 operate as a CGM controller device. Though the mobile device 104 is provided as an example of a device with which the CGM 102 communicates, the CGM 102 may communicate with other dedicated CGM controller devices for providing similar functionality that is described herein for the mobile device 104. In some cases, the CGM 102 processes the blood glucose data to provide an amount of glucose in interstitial fluid of the user 100. In some cases, the CGM 102 provides the blood glucose data to the mobile device 104 and/or pen device 103, and the mobile device 104 and/or pen device 103 processes the blood glucose data to manage the diabetic condition and provide treatment notifications as described herein.

In some embodiments, the blood glucose monitoring device is a flash glucose monitor (FGM) 111. The FGM 111 includes a subcutaneous sensor that is used to sense and monitor the amount of glucose in interstitial fluid of the user 100. A separate reader device, such as the mobile device 104, pen device 103, or another reader device, receives the blood glucose data from the sensor of the FGM 111 when the device is within range, such as but not limited to the RF range, of the sensor. The FGM 111 transmits an instantaneous blood glucose level or a graphical trend of the blood glucose level to the reader device for display. The mobile device 104 processes the blood glucose data to manage the diabetic condition and provide treatment notifications as described herein.

In some embodiments, the user 100 uses a blood glucose meter (BGM) 106 as a blood glucose monitoring device to monitor blood glucose levels. The BGM 106 includes a port 108 that receives a blood glucose measurement strip 110. The user 100 deposits a sample of blood on the blood glucose measurement strip 110. The BGM 106 analyzes the sample and measure the blood glucose level in the sample. The blood glucose level measured from the sample is displayed on a display 112 of the BGM 106 or communicated to an external device, such as the mobile device 104. The mobile device 104 processes the blood glucose data to manage the diabetic condition and provide treatment notifications as described herein.

The blood glucose level measured by the pen device 103 or BGM 106 or computed using data received from the CGM 102 or FGM 111, is used to treat the diabetic condition of the user 100. The mobile device 104 communicates with the CGM 102, FGM 111, the BGM 106, and/or pen device 103 using wired or wireless communications. The mobile device 104, the CGM 102, a CGM controller, the BGM 106, the FGM 111, or pen device 103 may be collectively referred to as user devices. The mobile device 104 communicates with the CGM 102, the FGM 111, the BGM 106, and/or pen device 103 using the same or different wireless protocols. For example, the mobile device 104 communicates with the CGM 102, FGM 111, the BGM 106, and/or pen device 103 using BLUETOOTH®, near field communication (NFC), THREAD®, WIFI®, ZIGBEE®, WI-MAX®, a cellular communication protocol, a proprietary wireless communication protocol, or another radio frequency (RF) communication protocol.

The mobile device 104 may receive data and store data for assisting in monitoring or treating the diabetic condition, such as, but not limited to optimizing and personalizing therapeutic protocols. The mobile device 104 may receive input from the user 100 via a user interface being provided on a display. The mobile device 104 may receive input via hard buttons or soft buttons provided on the display.

The mobile device 104 may be a connected smart device and/or may be in communication with other connected smart devices. Example connected smart devices may include a wearable device. The connected smart device may be an armband (e.g., a smart watch, such as an APPLE® watch, a FITBIT® armband, or other device capable of being worn on the arm of the user 100), a ring, glasses (e.g., GOOGLE® GLASS™), a headset (e.g., BLUETOOTH® headset), clothing (e.g., shirts, gloves, etc.), or another wearable device capable of being worn by the user 100. The connected smart device may be a wearable device or other device capable of monitoring the heart rate of the user 100, such as a heart rate monitor. The connected smart device may include a device capable of monitoring a wake/sleep state of the user, such as a wearable device, a heart rate monitor, a smart bed, or another device.

The mobile device 104 may be configured to determine information corresponding to the device's location (i.e., location information). For example, the mobile device 104 may be able to determine the geolocation (e.g., latitude and longitude) of the mobile device 104 using signals from a global positioning system (GPS) or triangulation via cellular communications. The mobile device 104 may communicate with the remote computing device 122 for accessing a third-party service (e.g., via an application programming interface (API)) to determine the location of the mobile device 104. The mobile device 104 may determine a relative location of the mobile device 104 via an indoor positioning technique by locating anchor nodes, such as WiFi access points and/or an RF beacon device 126, within a location that are communicably coupled to the mobile device 104. The RF beacon device 126 communicates a unique identifier via a short-range wireless communication, such as a BLUETOOTH® low energy (BLE) beacon or an NFC beacon. The mobile device 104 may receive the RF beacon and perform a lookup in a database (e.g., in information from the datastores 124) to determine a relative location associated with the unique identifier. For example, the mobile device 104 may determine that the RF beacon indicates that the device is in a particular building, type of building, room in a home or building, on a certain floor in a building, close to a predefined object, or is within the RF range of a beacon associated with another object or location.

Some embodiments of the mobile device 104 include one or more sensors for detecting a relative position of the device or information about the user 100. The mobile device 104 may detect a movement or a change in orientation. Based on the movement or change in orientation (or lack thereof) of the mobile device 104 over a period of time, the mobile device 104 may detect that the user 100 is standing, sitting, or lying down. In other words, the mobile device 104 detects/infers that the user 100 is awake. The mobile device 104 may detect that the user 100 is exercising when the movement or a change in orientation is greater than a threshold for a period of time. The mobile device 104 may detect the heartrate of the user 100 using a heartrate sensor. Based on the heartrate and/or the movement of the user 100 over a period of time, the mobile device 104 may detect whether the user 100 is asleep or awake. The information about the mobile device 104 or the user 100 is used to provide information about or treat the diabetic condition.

The mobile device 104 may provide information to the user 100 about the user's diabetic condition. For example, the mobile device 104 provides blood glucose levels, provides meal-related information, provides exercise-related information, provides treatment notifications, or generates graphs and other graphical user interfaces for display, or generates notifications that are provided to the user 100. The mobile device 104 may provide therapeutic protocol data to the pen device 103. For example, the mobile device 104 may provide insulin dose levels for an associated medication titration event (e.g., basal insulin titration event) to the pen device 103. Having received the insulin dose level, the pen device 103 may be configured to administer a corresponding amount of insulin.

The mobile device 104 may communicate with other devices directly via a wired communication and/or a short-range wireless communication (e.g., WI-FI®, BLUETOOTH®, BLE, NFC, or another suitable short-range wireless communication). The mobile device 104 may communicate indirectly with remote computing device(s) 122, or datastore(s) 124 via a network 120 (e.g., using a WI-FI® network, a cellular network, a WI-MAX® network, or another wired or wireless network). The network 120 is a wired or wireless network. The network 120 is used to communicate over the Internet to other devices.

The mobile device 104 may communicate with the remote computing device(s) 122 to generate user interfaces for display on the mobile device 104, perform remote computation, or to otherwise control a remote computing device. For example, the mobile device 104 may provide a user interface via an application (e.g., a web browser or other local application) that is generated locally for providing access to locally stored data or data from a remote computing device 122.

As described herein, the mobile device 104 may display information to the user 100 to assist in treatment of the user's diabetic condition. For example, as described herein, the mobile device 104 may provide information to the user 100 related to the user's health, devices assisting in treatment of the diabetic condition, the user's diabetic condition, and/or other information related to the user, the user's diabetic condition, and/or one or more devices to enable proper treatment of the diabetic condition. The mobile device 104 may provide blood glucose levels; provide meal-related information; provide exercise-related information; provide dosing information (e.g., dosing levels or reminders for glucose), provide information related to the state of one or more devices in the system (e.g., battery state, on/off state, error state, and/or other states related to the CGM 102, the FGM 111, the BGM 106, and/or one or more other devices); generate graphs and other graphical user interfaces for display, and/or generate alerts that may be provided to the user 100. The mobile device 104 may provide such information in the form of notifications (e.g., or alerts) to the user 100. The notifications may be provided to the user 100 via an application (e.g., diabetes-related application, exercise-related application, nutrition or mealtime-related application, another health-related application, or other type of application) residing locally thereon and/or via one or more remote computing devices 122. For example, one or more remote computing devices 122 may be implemented to execute the application for providing a graphical user interface with the notifications providing the information related to the diabetic condition and/or treatment thereof.

The mobile device 104 may allow the user 100 to interact with the application by receiving input from the user 100 and/or displaying information to the user. However, the user 100 may be prevented from interacting with the application when the mobile device 104 is in a locked state, which may prevent the user 100 from accessing certain information for enabling proper treatment of the diabetic condition. The mobile device 104 may provide a lock screen when the mobile device 104 is in the locked state. The locked state may be entered in response to the expiration of a timer or an input from the user 100. For example, the locked state may be entered into when the mobile device 104 goes into a sleep mode after an expiration of a sleep timer. The sleep mode may be enabled for battery savings on the mobile device 104 and the lock screen may be displayed when the mobile device 104 awakes from the sleep mode. The user 100 may access and/or interact with the application after receiving input from the user 100 that allows the mobile device 104 to operate in an unlocked or operational state. For example, the user 100 may input a security key and/or provide biometric data (e.g., fingerprint, facial features for facial recognition, and/or other biometric data) to authenticate the user 100 for unlocking the mobile device 104 and entering the unlocked or operational state. In the unlocked or operational state, the user 100 may provide input to the mobile device 104 for displaying certain information.

FIG. 1B is another perspective view of a representative user environment. As shown in FIG. 1B, the mobile device 104 that is operated by the user 100 may be displaying a current graphical user interface 105 for providing information to the user 100. For example, the mobile device may be in a locked state. While in the locked state, the mobile device 104 may display information 107 on a lock screen that is provided on the graphical user interface 105 of the mobile device 104. The mobile device 104 may display information 107 on the graphical user interface 105 as a home screen of the mobile device 104. For example, the mobile device 104 may display the information 107 on the graphical user interface 105 as a home screen after being unlocked and operate in an unlocked or operational state. The home screen may display one or more icons for applications that may be executed in response to a user actuation of the icons. The mobile device 104 may display information 107 on the graphical user interface 105 in response to the user 100 providing input to cause an application to be displayed on the mobile device. For example, the mobile device 104 may display the information 107 on the graphical user interface 105 after executing an application in response to a user actuation of an icon corresponding to the application on the mobile device. The mobile device 104 may display the information 107 on the graphical user interface 105 as a call screen that is provided when the mobile device is receiving an incoming call and/or displaying information to a user while making an outgoing call.

The information 107 may include information provided by the operating system on the mobile device 104 and/or one or more applications being executed by the mobile device in the background. The information 107 may include one or more overlay notifications 107a that are provided by applications executing in the background on the mobile device 104. In one example, the overlay notifications 107a may be lock screen notifications provided on the lock screen by applications executing in the background on the mobile device 104. In another example, the overlay notifications 107a may be home screen notifications provided on the home screen by applications executing in the background on the mobile device 104. Though the overlay notifications 107a may be described as being overlaid on a type of user interface or display, the overlay notifications 107a may be overlaid on a series of user interfaces. For example, the overlay notifications 107a may be overlaid on a lock screen of the mobile device and continue to be overlaid on a home screen, call screen, or other application that is executing in the foreground of the mobile device 104.

The overlay notifications 107a may be displayed in response to one or more overlay notification triggering events at the mobile device 104. For example, the overlay notifications 107a may be displayed in response to one or more messages received from one or more devices, such as one or more of the remote computing devices 122, the RF beacon device 126, the BGM 106, insulin pumps 116, 118, and/or one or more devices. The overlay notifications 107a may be displayed in response to a local generation of the overlay notifications 107a by an application residing and executing locally on the mobile device 104. The overlay notifications 107a may be displayed in response to an actuation of a button 109 on the mobile device 104. For example, when the mobile device 104 is in a sleep state, the mobile device 104 may awake in response to the actuation of the button 109 or another overlay notification triggering event and display the overlay notifications 107a for a period of time or until receiving an input from the user 100.

As multiple applications may be operating on the mobile device 104, and/or each application may generate multiple notifications that may be displayed at the same time, the user 100 may receive many notifications over a period of time. The notifications that are provided may be redundant, ignored, and/or inconvenient to the user 100. As the notifications that are related to the health of the user 100 may be of a higher priority or importance to the user 100 than other information provided as notifications (e.g., push notifications) to the user, and/or provided in order to treat a medical condition (e.g., the user 100's diabetic condition), the health-related notifications may be inadvertently ignored or hidden by other notifications. At times, the number of notifications (e.g., lock screen notifications) may be provided in a scrollable list, which may cause the user 100 to have to scroll through to identify the important notifications for enabling treatment of the medical condition (e.g., the user 100's diabetic condition).

Even when one or a few applications are operating on the mobile device 104 for enabling treatment of the medical condition (e.g., the user 100's diabetic condition), multiple notifications may be displayed to assist in treatment of the medical condition (e.g., the user 100's diabetic condition). The application or applications may still cause the generation of many notifications to be provided over a period of time. Thus, even when the user 100 is sifting through notifications related to a particular application or applications for information related to the user 100's health (e.g., diabetes-related application, exercise-related application, nutrition or mealtime-related application, another health-related application, or other type of application), the user 100 may inadvertently ignore important information (e.g., information related to blood-glucose levels trending or going above or below a threshold, notifications related to the user 100 finding or ingesting carbohydrates, and/or other important information related to treatment of the medical condition).

The limited screen space on the mobile device 104 may limit the amount of information that is provided to the user at a given time. As described herein, the user 100 may have to scroll through multiple notifications to access additional information when such information is unable to be provided on a single screen. For example, the user 100 may have to scroll through notifications provided on the lock screen to access additional notifications, which may be related to medical conditions or the health of the user 100. This may cause the user 100 to miss certain types of information and/or alerts that may be important for treatment of the medical condition (e.g., the user 100's diabetic condition), or view information at different times, which may cause problems in the treatment of the medical condition.

The overlay notifications 107a being overlayed over one or more user interfaces (e.g., one or a series of user interfaces, as described herein) may allow the user 100 to view important information related to a medical condition or health of the user 100. For example, the overlay notifications 107a being overlayed over the lock screen to treat the medical condition (e.g., the user 100's diabetic condition) may allow the user 100 to use the mobile device 104 to treat the diabetic condition without unlocking the mobile device 104 and viewing information and/or alerts via an application. As such, the mobile device 104 may be used to treat the medical condition (e.g., the user 100's diabetic condition), while also preserving battery life by utilizing a sleep mode and/or preserving the security of the user 100 by locking the mobile device 104 to prevent other users from accessing personal health information and/or other personal or valuable information of the user 100. The overlay notifications 107a being overlayed over other user interfaces, such as the home screen, the call screen, or another user interface displayed by the operating system or an application on the mobile device 104 may allow the user 100 to treat the medical condition (e.g., the user 100's diabetic condition), even while one or more applications from which the information is being provided via the overlay notifications 107a is operating in the background of the mobile device 104.

FIG. 1C is another perspective view of a representative user environment. As shown in FIG. 1C, the mobile device 104 that is operated by the user 100 may display an overlay notification 107b when operating in the locked state. Examples are provided herein for providing the overlay notification 107b as a diabetic overlay notification. However, other types of medical overlay notifications may similarly be provided for providing information related to and/or enabling treatment of other medical conditions, as described herein. The overlay notification 107b may be provided as a lock screen notification, a home screen notification, or another notification that is overlaid over another graphical user interface being displayed to the user of the mobile device or another computing device, as described herein.

The overlay notification 107b may be dynamically displayed on the graphical user interface 105 that is currently displayed on the mobile device 104. The overlay notification 107b may be dynamically and/or continuously updated on the graphical user interface 105 of the mobile device 104 in response to updated information. The overlay notification 107b may be generated and/or displayed by an application (e.g., diabetes-related application, exercise-related application, nutrition or mealtime-related application, another health-related application, or other type of diabetes application) residing locally on the mobile device 104 and/or via information from one or more other devices. For example, when the mobile device 104 is in a sleep state, the mobile device 104 may awaken in response to an overlay notification triggering event and display the overlay notifications 107b for a period of time or until receiving another overlay notification triggering event.

In one example, the overlay notification 107b, which may be a medical overlay notification or health-related overlay notification, may be displayed as a Continuously Updated Event Monitoring Entity (CUE-ME) notification. The CUE-ME notification may include a dynamically and/or continuously updated notification. The CUE-ME notification may be dynamically updated based on data being monitored at the mobile device 104 and/or one or more other devices (e.g., the insulin pump 116, 118, the CGM 102, the BGM 106, the FGM 111, the pen device 103, etc.). The data may include medical data or data related to the health of the user 100. For example, the overlay notification 107b may include a CUE-ME notification that is dynamically and/or continuously updated with the data indicating a current blood-glucose level of the user 100 and/or other information related to a diabetic condition of the user 100.

In one example, the CUE-ME notification may be displayed as a Live Activity functionality in an iOS operating system on the mobile device 104. The local application (e.g., diabetes-related application, exercise-related application, nutrition or mealtime-related application, another health-related application, or other type of application) residing on the mobile device 104 may make function calls to and/or application programming interface (API) calls to the operating system on the mobile device 100 and/or another service (e.g., service executing on the one or more remote computing devices 122) to display the CUE-ME notification as a push notification on the lock screen that is dynamically updated based on changes in data. The user 100 may monitor changes and/or progress of health-related events and/or other events from the lock screen of the mobile device 104 without unlocking the mobile device 104, finding the app, and/or bringing app to the foreground of the mobile device 104. The CUE-ME notification may be dynamically updated to give the user 100 continuous updates and details about a certain event or events without generating separate notifications. Though the iOS and the Live Activities functionality of the iOS are provided as examples, similar functionality may be implemented on devices operating with other operating systems and/or services, such as an ANDROID operating system or another operating system or service.

In one example, the CUE-ME notification may be displayed as a Dynamic Island functionality in an iOS operating system on the mobile device 104. The local application (e.g., diabetes-related application, exercise-related application, nutrition or mealtime-related application, another health-related application, or other type of application) residing on the mobile device 104 may make function calls to and/or application programming interface API calls to the operating system on the mobile device 100 and/or another service (e.g., service executing on the one or more remote computing devices 122) to display the CUE-ME notification as a push notification on the home screen that is dynamically updated based on changes in data. The user 100 may monitor changes and/or progress of health-related events and/or other events from the home screen of the mobile device 104 without finding the app and/or bringing the app to the foreground of the mobile device 104. The CUE-ME notification may be dynamically updated to give the user 100 continuous updates and details about a certain event or events without generating separate notifications. Though the iOS and the Dynamic Island functionality of the iOS are provided as examples, similar functionality may be implemented on devices operating with other operating systems and/or services, such as an ANDROID operating system or another operating system or service.

The overlay notification 107b may be provided on the mobile device 104 in response to one or more overlay notification triggering events at the mobile device 104. The overlay notification triggering event may be based on data related to a medical condition of the user 100 (e.g., the user's diabetic condition). The overlay notification 107b may be dynamically updated as a CUE-ME notification based on data being monitored by one or more devices (e.g., the mobile device 104, the insulin pump 116, 118, the CGM 102, the BGM 106, the FGM 111, the pen device 103, remote computing device 122, etc.). The overlay notification 107b may be displayed and/or continuously updated for a period of time or until an overlay notification removal event is detected. The period of time may be configured to be a predefined period of time from the overlay notification triggering event. The overlay notification removal event may be based on data indicating an end to an event or may be determined to be a time period after an overlay notification triggering event.

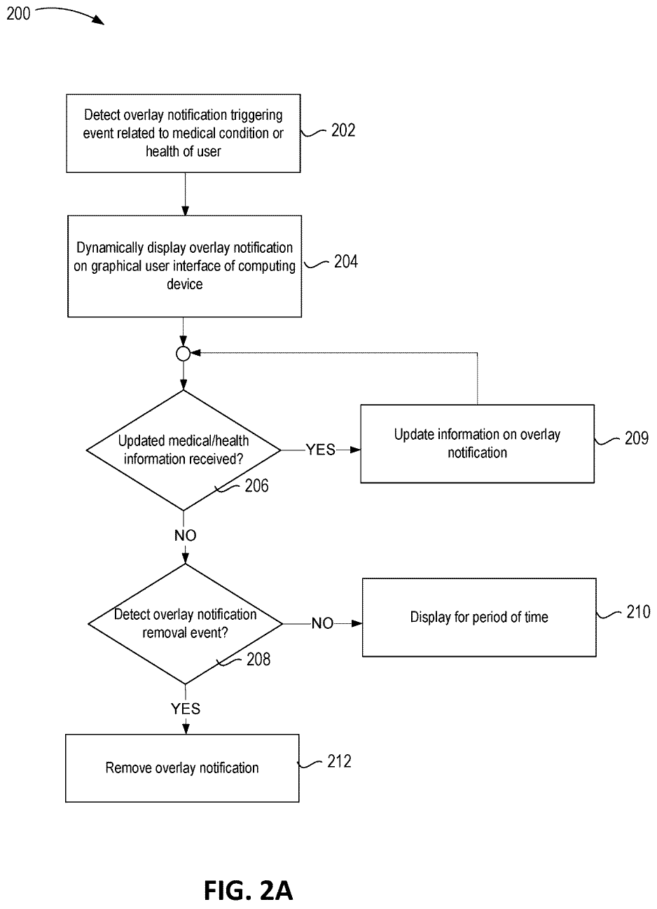

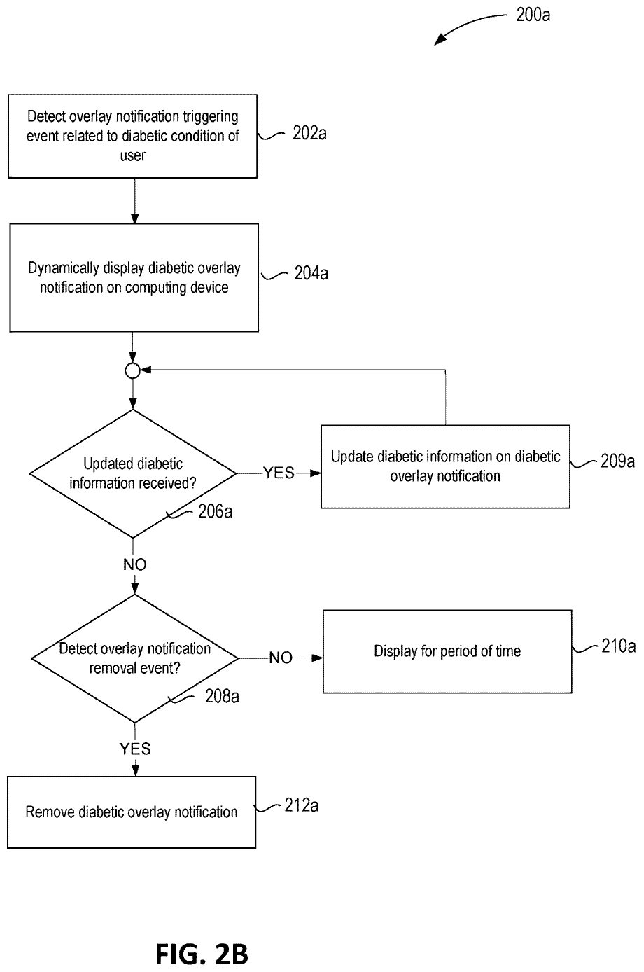

FIG. 2A shows a flowchart of an example process 200 for displaying, updating, and/or removing overlay notifications, which may be provided as CUE-ME notifications. One or more portions of the process 200 may be performed by one or more computing devices. For example, the one or more portions of the process 200 may be performed by one or more mobile devices, such as mobile device 104 shown in FIGS. 1A-1C, and/or one or more remote computing devices, such as remote computing device 122 shown in FIGS. 1A-1C. One or more portions of the process 200 may be stored in memory as computer-readable or machine-readable instructions that may be executed by a processor of the one or more computing devices. Though portions of the process 200 may be described herein as being performed by a particular computing device, the process 200 may be performed by another computing device or distributed across multiple computing devices, such as one or more mobile devices, remote computing devices, and/or one or more other devices.

As illustrated in FIG. 2A, a computing device detects an overlay notification triggering event at 202. The overlay notification triggering event may be related to a medical condition or health of a user. The computing device may receive data related to a condition of the user from one or more devices (e.g., the mobile device 104, the insulin pump 116, 118, the CGM 102, the BGM 106, the FGM 111, the pen device 103, etc., as shown in FIGS. 1A-1C). The received data may include an indication of chronic conditions for which an alert may be provided. The chronic conditions, for example, may comprise abnormal glucose levels, high blood pressure, abnormal heart beats per minute (bpm), and/or other chronic conditions.

In one example, the overlay notification triggering event may be detected, at 202, based on an indication that the user's blood glucose is above or below a glucose threshold or range and the overlay notification may provide current blood glucose level information for the user to monitor the blood glucose level until it is back in range or above/below the glucose threshold. The overlay notification triggering event may be detected based on an indication of an abnormal increase or decrease of blood glucose level within a predefined period of time. The glucose level of the user may accelerate with an abnormal rate of change, for example, glucose rising too quickly or glucose falling too quickly within some period of time (e.g., 15 min) regardless of the current glucose level of the user (e.g., even if they are normoglycemic or not yet in a hypoglycemic or hyperglycemic state). The overlay notification triggering event may be detected based on an indication of a predictive hypoglycemic event or a predictive hyperglycemic event, which means the blood glucose levels may be predicted to be hyperglycemic or hypoglycemic within some period of time from the current time based on the amount the blood glucose levels are rising or falling within a period of time.

The overlay notification triggering event may be detected, at 202, based on an indication of a performance of exercise by the user or an exercise event performed by the user. For example, the overlay notification triggering event may be detected based on an indication that a breathing rate, a heart rate, or a blood flow of a user is above and/or below a predefined threshold or within a predefined range, which may be detected by the computing device (e.g., mobile device and/or other devices, such as wearable devices). The overlay notification triggering event may be detected based on an indication of a timeframe or time at which an exercise event is scheduled (e.g., in a digital calendar of events). The overlay notification triggering event may be detected based on an input received by the user, such as an indication of a time or timeframe at which an exercise event is entered in a logbook (e.g., in an application). The overlay notification triggering event may be detected based on an indication that the activity level of the user is above a predefined threshold or within a predefined range (e.g., as measure by the accelerometer, gyroscope, and/or one or more other sensors on the mobile device and/or one or more other devices described herein).

The overlay notification triggering event may be detected, at 202, based on an indication of a mealtime, snack, or meal being consumed by the user. For example, the overlay notification triggering event may be detected based on an indication that the user has consumed a meal or snack (e.g., a meal or post-meal snack). The indication may be a timeframe or time at which the meal or snack is scheduled (e.g., in a digital calendar of events or a predefined time). The indication may be a timeframe or time at which carbohydrates are logged into a logbook (e.g., in an application). The indication may also be based on a glucose level being above or below a monitored glucose level for a predefined period of time (e.g., up to 2-3 hours after a mealtime or entering carbs into logbook).

The overlay notification triggering event may be detected, at 202, based on an indication that user has received insulin. For example, the overlay notification triggering event may be detected in response to an indication that the user has received a predefined amount of short-acting insulin within a predefined period of time. The data indicating the insulin injections may be received from the insulin pump 116, 118, the pen device 103, and/or another device, as shown in FIGS. 1A-1C. The overlay notification triggering event may be detected in response to an entry of bolus insulin into a logbook in a local application. The overlay notification triggering event may include an indication that a time period has elapsed after entering bolus insulin into a logbook (e.g., in an application).

The overlay notification triggering event may be detected, at 202, based on indications of additional, or alternative, user input into a logbook or a period of time after logging a condition in the logbook (e.g., in an application). For example, described elsewhere herein are examples of logging an entry of bolus insulin into the logbook, logging carbohydrates or a meal into the logbook, and/or logging an exercise event into the logbook. Other events that may be logged into the logbook for generating an overlay notification triggering event may include a stress-related event, an illness-related event, and/or an event related to taking medication.

The overlay notification may be related to a state or status of one or more devices that may be implemented in treatment or assistance with treatment of the medical condition (e.g., the user's diabetic condition). For example, the overlay notification triggering event for the overlay notification may be detected, at 202, based on an indication of pump malfunctions (e.g., pump error, such as an occlusion, a mechanical error, an electronic error, and/or another pump malfunction) received from the insulin pump (e.g., insulin pump 116, 118 shown in FIGS. 1A-1C). The overlay notification triggering event may be detected based on an indication of CGM malfunctions (e.g., a mechanical error, an electronic error, and/or another CGM malfunction) received from the CGM (e.g., CGM 102 shown in FIGS. 1A-1C). The overlay notification triggering event may be detected based on an indication of BGM malfunctions (e.g., a mechanical error, an electronic error, an error reading the blood glucose measurement strip, and/or another BGM malfunction) received from the BGM (e.g., BGM 106 shown in FIGS. 1A-1C). The overlay notification triggering event may be detected based on an indication of FGM malfunctions (e.g., a mechanical error, an electronic error, and/or another FGM malfunction) received from the FGM (e.g., FGM 111 shown in FIGS. 1A-1C). The overlay notification triggering event may be detected based on an indication of a malfunction (e.g., a mechanical error, an electronic error, and/or another malfunction with the pen device) received from the pen device (e.g., pen device 103 shown in FIGS. 1A-1C).

The overlay notification may be displayed in response to one or more other indications or messages received from one or more devices (e.g., one or more of the remote computing devices 122, the RF beacon device 126, the BGM 106, insulin pumps 116, 118, and/or one or more other devices shown in FIGS. 1A-1C). Based on the message type and/or information in the message, the overlay notification may be triggered.

The overlay notification triggering event may be detected, at 202, based on a recognition, by the computing device, of one or more other devices that may be implemented in treatment or assistance with treatment of the medical condition (e.g., the user's diabetic condition). For example, the overlay notification triggering event may include an indication that another device has paired with the computing device, or that a period of time has passed after pairing of the device (e.g., the insulin pump 116, 118, the CGM 102, the BGM 106, the FGM 111, the pen device 103, etc. shown in FIGS. 1A-1C). The pairing may be performed by an exchange of information and/or keys to register and/or associate the devices in memory for enabling communication. The overlay notification triggering event may be detected based on an indication that a sensor warmup time has lapsed after pairing of the sensor (e.g., for the CGM 102, the FGM 111, the BGM 106, etc. as shown in FIGS. 1A-1C). When a sensor (e.g., for the CGM 102, the FGM 111, the BGM 106, etc. as shown in FIGS. 1A-1C) is implanted in the body of the user or otherwise paired or connected to the body of the user, the sensor may utilize a sensor warmup time to get acclimated for getting used to the body of the user and in order to provide accurate sensor readings and provide accurate information from the sensor readings.

The overlay notification triggering event may be detected, at 202, based on location data (e.g., geolocation data) for certain pre-defined and/or user-defined locations (e.g., a restaurant, a gym, etc.). The overlay notification triggering event may be detected based on predefined and/or user defined categories of locations (e.g., cinemas, schools, etc.). The computing device may utilize an application programming interface (API) of a location service (e.g., operating on one or more remote computing devices) to receive information associated with a particular location, such as an identifier of the location name, location category, location address, and/or other information associated with the location. The location information may be compared to the pre-defined and/or user-defined locations/categories. A triggering event for the overlay notification may be generated when the location information matches or is determined to be associated with the pre-defined and/or user-defined locations/categories.

Though predefined times and/or timeframes are described herein for which the overlay notification triggering event may be detected at 202, an overlay notification triggering event may be detected at other times and/or timeframes. For example, the user may set a predefined time or timeframe at which the overlay notification may be displayed. The predefined time or timeframe may be recurring, such as each day, each week, each month, etc. The predefined time or timeframe may be associated with a particular event, such as a meeting, a movie, while driving, and/or while performing another event. The events may be detected by the computing device from an entry in a digital calendar, a detection of a location identifier that indicates a location (e.g., GPS coordinates associated with a movie theater, the computing device pairing to a vehicle having a vehicle identifier, etc.). The overlay notification may be triggered when the user is determined to be asleep or be awakening from being asleep, such as when the user is scheduled to be or detected to be sleeping and/or is detected to be awoken (e.g., based on the sensors in the computing device). This may allow the user to wake up and check the information being displayed on the lock screen without having to unlock the computing device, or check the information on the home screen or screen of another application on which the overlay notification is being overlaid without having to open the native application on the mobile device. The information being provided may be time sensitive and the user may need to be aware of the information for treating their medical condition quickly to prevent an extreme diabetic condition from occurring or worsening.

The overlay notification triggering event may be a manual triggering event. For example, the overlay notification triggering event may include a user input in the form of an actuation of a button (e.g., button 109 on the mobile device 104 shown in FIGS. 1A-1C) or receipt of input on the display of the computing device. The manual triggering event initiated by the user may include a medical condition, a health-related condition, or a subjective concern or curiosity of his own health condition. The overlay notification may be displayed for a predefined period of time after the user input is received or until an overlay notification removal event is detected.

Different overlay notification triggering events may trigger the same or different overlay notifications. The overlay notification may be provided in response to a single overlay notification triggering event, or a combination of one or more triggering events, as described herein.

At 204, the computing device may dynamically or statically display an overlay notification on the graphical user interface being displayed (e.g., lock screen, home screen, call screen, and/or other graphical user interface) on the computing device. In one example, when the computing device detects that an overlay notification is to be displayed, the computing device may display the overlay notification, which may be a medical overlay notification or other health-related overlay notification, as a CUE-ME notification that is dynamically and/or continuously updated in response to updated medical information and/or health-related information. For example, the overlay notification may be dynamically updated based on data being monitored at one or more devices (e.g., mobile device 104, the insulin pump 116, 118, the CGM 102, the BGM 106, the FGM 111, the pen device 103, etc., as shown in FIGS. 1A-1C).

When the computing device is in a sleep mode, the computing device may receive a wake indicator before displaying the overlay notification (e.g., as a lock screen notification on the lock screen or a home screen notification on the home screen). The wake indicator may wake the computing device from a sleep state and the overlay notification may be displayed in response to the overlay notification triggering event. The wake indicator may be detected as part of the overlay notification triggering event, or a separate indication altogether (e.g., in the same or different message). The wake indicator and/or the overlay notification triggering event may be received in one or more messages from other devices. The overlay notification may comprise a textual notification and/or other information to the user, as described herein.

The overlay notification may continue to be overlaid over a series of graphical user interfaces displayed on the computing device. For example, the overlay notification may be overlaid on the lock screen as a lock screen notification and continue to be overlayed on the home screen as a home screen notification, and/or continue to be overlaid on one or more other applications being displayed on the computing device. The computing device may determine how and where to display the overlay notification based on the graphical user interface over which the overlay notification is being displayed. For example, the size, shape, and/or location of the overlay notification may be different depending on the graphical user interface over which the overlay notification is being displayed.

At 206, the computing device may determine whether updated data has been received that includes information for updating the overlay notification. The data may include medical information or other information related to the health of the user. If updated data has been received that includes information for updating the overlay notification, the computing device may update the information on the overlay notification at 209. For example, the overlay notification may include a CUE-ME notification that is dynamically updated with the data indicating a current blood-glucose level of the user and/or other information related to a diabetic condition or other medical condition of the user. In order for the overlay notification to be updated, the information may be changed by at least a threshold. For example, the blood glucose level of the user may be changed by a threshold in order to update the information on the overlay notification.

The overlay notification may be displayed for a period of time or until another overlay notification removal event is detected at 208. If the computing device determines that the overlay notification has been displayed for a predefined period of time (e.g., without receiving another overlay notification removal event and/or another user input interacting with the overlay notification), the computing device may display the overlay notification for the period of time at 210. The period of time may be similar to and/or coincide with the period of time of the sleep timer for the computing device entering the sleep state. The period of time may be configured to be a predefined period of time from the overlay notification triggering event.

Though predefined times and/or timeframes are described herein for which the overlay notification may be removed, an overlay notification removal event may be detected at other times and/or timeframes. For example, the user may set a predefined time or timeframe at which the overlay notification may be removed (e.g., after being displayed). The overlay notification may be removed when the computing device enters the sleep state. Different overlay notification removal events may be associated with a particular event and/or overlay notification triggering event. Different overlay notification removal events may be associated with the same or different predefined periods of time. The predefined period of time may be a period of time without input or interaction from the user with the computing device and/or the overlay notification. If the user provided input to the computing device while the overlay notification is displayed, the predefined period of time may be reset. Provided below in TABLE 1 are example predefined periods of time for detecting an overlay notification removal event after detection of a corresponding overlay notification triggering event:

TABLE 1

| | |
|---|---|
| Triggering events | Predefined Periods of Time |

| Hypo (Blood Glucose) | 25 | minutes |
|---|---|---|
| Hyper (Blood Glucose) | 25 | minutes |
| Sleep State Ends | Period of sleep timer | |
| Log (perform) exercise | 3 hours after logging exercise | |
| Log (eat) carbs | 3 hours after logging carbs | |
| Log (deliver) bolus | 3 hours after logging bolus | |
| Log stress | 8 | hours |
| Log illness | 8 | hours |
| Log medication | 8 | hours |
| Feeling concerned or curious | 8 | hours |
| (e.g., input from user) | | |

| | |
|---|---|
| Monitoring countdown time | 2 hours sensor warmup time |
| Monitoring countdown time | 2 hours until sensor expires |

In another example, the overlay notification may be persistently displayed on the lock screen. For example, the computing device may receive an input from the user in the settings associated with the overlay notification to persistently display the overlay notification, or a button for accessing information via the overlay notification, when a graphical user interface (e.g., a lock screen, a home screen, call screen, and/or other graphical user interface) is being displayed.

The computing device may detect an overlay notification removal event at 208. If an overlay notification removal event is detected at 208, the overlay notification, which may be a medical overlay notification or other health-related overlay notification, may be removed from the graphical user interface being displayed (e.g., lock screen, home screen, call screen, and/or other graphical user interface) and/or display of the computing device at 212. The overlay notification removal event may be based on data related to the diabetic condition or another medical condition. An example overlay notification removal event may be detected based on an indication that an extreme diabetic state (e.g., hypoglycemic state or hyperglycemic state) has ceased and/or an indication that the user's blood-glucose level has returned to a normal diabetic state (e.g., for a predefined period of time). For example, the overlay notification removal event may be detected based on an indication that the user's blood glucose is above or below a glucose threshold or within an acceptable range. The overlay notification removal event may be detected based on an indication that an abnormal increase or decrease of the user's blood glucose level has ceased to a stable or normal range of increases/decreases (e.g., for a predefined period of time).

The overlay notification removal event may be detected based on an indication that an exercise event performed by the user has ceased (e.g., for a predefined period of time). For example, the overlay notification removal event may be detected based on an indication that a breathing rate, a heart rate, or a blood flow of a user has returned to below a predefined threshold or within another predefined range. The overlay notification removal event may be detected based on an indication of a time at which an exercise event is scheduled to end (e.g., in a digital calendar of events). The overlay notification removal event may be detected based on an indication that the activity level of the user is below a predefined threshold or within a predefined range, which may be lower than the range of the overlay notification triggering event (e.g., as measure by the accelerometer, gyroscope, and/or one or more other sensors on the computing device and/or one or more other devices described herein).

The overlay notification removal event may be detected based on an indication that a mealtime has ended or a predefined period of time has elapsed after the start of a mealtime. For example, the overlay notification removal event may be detected based on an indication that the user has finished a meal or snack (e.g., a meal or post-meal snack). The indication may be a time at which the meal or snack is scheduled to end (e.g., in a digital calendar of events or a predefined time). The indication may be a time at which carbohydrates are logged into a logbook (e.g., in an application). The indication may also be based on a glucose level rising above a threshold.

The overlay notification removal event may be detected based on an indication that a predefined period of time has elapsed since the user has received insulin or entered bolus insulin into a logbook (e.g., in an application). The data indicating the insulin injections may be received from an insulin pump (e.g., insulin pump 116, 118, as shown in FIGS. 1A-1C), a pen (e.g., pen device 103, as shown in FIGS. 1A-1C), and/or another device. The overlay notification removal event may be detected based on an indication that a period of time has elapsed since additional, or alternative, user input has been entered into a logbook or other conditions have been logged in the logbook (e.g., in an application).

The overlay notification removal event may be detected based on an indication that a predefined period of time has elapsed since a device malfunction or a message has been received indicating a device malfunction (e.g., such as pump malfunctions received from the insulin pump 116, 118, CGM malfunctions received from the CGM 102, BGM malfunctions received from the BGM 106, FGM malfunctions received from the FGM 111, and/or malfunctions received from the pen device 103, as shown in FIGS. 1A-1C).

The overlay notification may be removed in response to one or more other indications or messages received from one or more devices (e.g., such as one or more of the remote computing devices 122, the RF beacon device 126, the BGM 106, insulin pumps 116, 118, and/or one or more other devices, as shown in FIGS. 1A-1C). Based on the message type and/or information in the message, the overlay notification may be removed.

The overlay notification may be removed a predefined period of time after the indication that another device has paired with the computing device. The overlay notification removal event may be detected after the predefined period of time has lapsed after pairing of a sensor device (e.g., the CGM 102, the FGM 111, the BGM 106, etc.) or another device with the computing device.

The overlay notification may be removed based on a location data (e.g., geolocation data) associated with certain pre-defined and/or user-defined locations (e.g., a restaurant, a gym, etc.). The overlay notification may be removed based on predefined and/or user defined categories of locations (e.g., cinemas, schools, etc.). Location information indicating a location of the computing device may be compared to the pre-defined and/or user-defined locations/categories. The overlay notification may be removed when the location information indicating the location of the computing device leaves a location indicated in the pre-defined and/or user-defined locations/categories. For example, the overlay notification may be removed when the location information fails to match or be associated with the pre-defined and/or user-defined locations/categories.

The overlay notification removal event detected for removal of the overlay notification may be a manual triggering event. For example, the overlay notification removal event may include a user input in the form of an actuation of a button on the computing device or receipt of input on the display of the computing device.

One or more overlay notification triggering events may be configured to be manually turned on or turned off Similarly, one or more overlay notification removal events may be configured to be manually turned on or off The user may turn on or turn off any manually or automatically configured overlay notification triggering events and/or overlay notification removal events. This may allow the user to activate the events for enabling/disabling the overlay notifications and store the activated events in memory.

The overlay notification triggering event and/or the overlay notification removal event may be manually or automatically configured. For example, the user may manually type in or select (e.g., from a predefined menu) a specific triggering event and/or removal event associated with predefined glucose level thresholds in the computing device to configure it as the display and/or removal of the overlay notification. The computing device may also, or alternatively, configure certain predefined triggering events and/or removal events (e.g., predefined medical condition triggering events, timers, etc.).

The user may unlock the lock screen and/or display the application (e.g., diabetes-related application, exercise-related application, nutrition or mealtime-related application, another health-related application, or other type of application) residing locally on the computing device in response to a selection of the overlay notification. For example, the user may unlock the lock screen on the computing device and/or remove the overlay notification by displaying a diabetes application, if the computing device receives a selection of the overlay notification, or a predefined button thereon. The local application (e.g., diabetes-related application, exercise-related application, nutrition or mealtime-related application, another health-related application, or other type of application) may be brought to the foreground on the display of the computing device to allow for the user to access additional features and/or information to enable treatment of the user's medical condition. The local application being brought to the foreground on the display of the computing device may also, or alternatively, allow the user to interact with other devices (e.g., paired devices) through the computing device. The local application may occupy a greater amount of space on the display of the computing device than previously occupied by the overlay notification. The user may be allowed to adjust parameters for controlling other devices through the local application based on the information previously displayed on the overlay notification. For example, the user may access the local application and select a button on the computing device to provide medication to the user (e.g., via one or more messages transmitted from the computing device) and/or adjust thresholds for enabling treatment of the medical condition of the user.

The overlay notification may be updated dynamically, as described herein, based on updated data to provide continuous updating to reduce a number of notifications sent or displayed to a user, while also making information readily accessible and available for monitoring and/or treating a diabetic condition. FIG. 2B shows a flowchart of an example process 200a for displaying and/or removing diabetic overlay notifications for a diabetes-related application. One or more portions of the process 200a may be performed by one or more computing devices. For example, the one or more portions of the process 200a may be performed by one or more mobile devices, such as mobile device 104 shown in FIGS. 1A-1C, and/or one or more remote computing devices, such as remote computing device 122 shown in FIGS. 1A-1C. One or more portions of the process 200a may be stored in memory as computer-readable or machine-readable instructions that may be executed by a processor of the one or more computing devices. Though portions of the process 200a may be described herein as being performed by a particular computing device, the process 200a may be performed by another computing device or distributed across multiple computing devices, such as one or more mobile devices, remote computing devices, and/or one or more other devices.

As illustrated in FIG. 2B, at 202a, the computing device may detect an overlay notification triggering event related to a diabetic condition of the user. The computing device may receive data related to a diabetic condition of a person from one or more devices (e.g., the mobile device 104, the insulin pump 116, 118, the CGM 102, the BGM 106, the FGM 111, the pen device 103, etc., as shown in FIGS. 1A-1C) The received data may include an indication of chronic conditions for which an alert may be provided. The chronic conditions, for example, may comprise abnormal glucose levels, high blood pressure, abnormal heart beats per minute (bpm), etc. For example, abnormal glucose levels may be glucose levels that are either higher than a predefined upper threshold, or lower than a predefined lower threshold.

The overlay notification triggering event may be detected based on an indication of an extreme diabetic state (e.g., hypoglycemic state or hyperglycemic state). For example, the overlay notification triggering event may be detected based on an indication that the user's blood glucose is above or below a glucose threshold or range and the overlay notification may provide current blood glucose level information for the user to monitor the blood glucose level until it is back in range or above/below the glucose threshold. The overlay notification triggering event may be detected based on an indication of an abnormal increase or decrease of blood glucose level within a predefined period of time. The glucose level of the user may accelerate with an abnormal rate of change, for example, glucose rising too quickly or glucose falling too quickly within some period of time (e.g., 15 min) regardless of the current glucose level of the user (e.g., even if they are normoglycemic or not yet in a hypoglycemic or hyperglycemic state). The overlay notification triggering event may be detected based on an indication of a predictive hypoglycemic event or a predictive hyperglycemic event, which means the blood glucose levels may be predicted to be hyper or hypo within some period of time from the current time based on the amount the blood glucose levels are rising or falling within a period of time.

The overlay notification triggering event may be detected based on an indication that user has received insulin. For example, the overlay notification triggering event may include an indication that the user has received a predefined amount of short-acting insulin within a predefined period of time. The data indicating the insulin injections may be received from the insulin pump 116, 118, the pen device 103, and/or another device, as shown in FIGS. 1A-1C. The overlay notification triggering event may include an indication that a time period has elapsed after entering bolus insulin into a logbook (e.g., in an application).

The diabetic overlay notification may be displayed in response to one or more other indications or messages received from one or more devices (e.g., one or more of the remote computing devices 122, the RF beacon device 126, the BGM 106, insulin pumps 116, 118, and/or one or more other devices shown in FIGS. 1A-1C). Based on the message type and/or information in the message, the diabetic overlay notification may be triggered.

Different overlay notification triggering events may trigger the same or different diabetic overlay notifications. The diabetic overlay notification may be provided in response to a single overlay notification triggering event, or a combination of one or more triggering events, as described herein.

At 204a, the computing device may dynamically display a diabetic overlay notification on the graphical user interface being displayed (e.g., lock screen, home screen, call screen, and/or other graphical user interface) on the computing device. In one example, when the computing device detects that a diabetic overlay notification is to be displayed on a lock screen, the computing device may display the diabetic overlay notification (e.g., or other medical overlay notification) as a CUE-ME notification that is dynamically updated in response to updated information related to the diabetic condition of the user. For example, the diabetic overlay notification may be dynamically updated based on data being monitored at the computing device (e.g., mobile device 104, the insulin pump 116, 118, the CGM 102, the BGM 106, the FGM 111, the pen device 103, etc., as shown in FIGS. 1A-1C).

At 206a, the computing device may determine whether updated data related to the diabetic condition of the user has been received that includes information for updating the diabetic overlay notification (e.g., or other medical overlay notification). If updated data has been received that includes information for updating the diabetic overlay notification, the computing device may update the information related to the diabetic condition on the overlay notification at 209a. For example, the diabetic overlay notification may include a CUE-ME notification that is dynamically updated with the data indicating a current blood-glucose level of the user and/or other information related to a diabetic condition of the user.

The diabetic overlay notification may be displayed for a period of time or until another overlay notification removal event is detected at 208a. If the computing device determines that the overlay notification has been displayed for a predefined period of time (e.g., without receiving another overlay notification removal event and/or another user input interacting with the diabetic overlay notification), the computing device may display the diabetic overlay notification for the period of time at 210a. The period of time may be similar to and/or coincide with the period of time of the sleep timer for the computing device entering the sleep state. The period of time may be configured to be a predefined period of time from the overlay notification triggering event.

Though predefined times and/or timeframes are described herein for which the diabetic overlay notification may be removed, an overlay notification removal event may be detected at other times and/or timeframes. For example, the user may set a predefined time or timeframe at which the diabetic overlay notification may be removed (e.g., after being displayed). The diabetic overlay notification may be removed when the computing device enters the sleep state. Different overlay notification removal events may be associated with a particular event and/or overlay notification triggering event. Different overlay notification removal events may be associated with the same or different predefined periods of time. The predefined period of time may be a period of time without input or interaction from the user with the computing device and/or the diabetic overlay notification. If the user provided input to the computing device while the diabetic overlay notification is displayed, the predefined period of time may be reset.

The computing device may detect an overlay notification removal event at 208a. If the overlay notification removal event is detected at 208a, the diabetic overlay notification may be removed from the graphical user interface being displayed (e.g., lock screen, home screen, call screen, and/or other graphical user interface) and/or display of the computing device at 212a. The overlay notification removal event may be based on data related to the diabetic condition of the user. An example overlay notification removal event may be detected based on an indication that an extreme diabetic state (e.g., hypoglycemic state or hyperglycemic state) has ceased and/or an indication that the user's blood-glucose level has returned to a normal diabetic state (e.g., for a predefined period of time). For example, the overlay notification removal event may be detected based on an indication that the user's blood glucose is above or below a glucose threshold or within an acceptable range. The overlay notification removal event may be detected based on an indication that an abnormal increase or decrease of the user's blood glucose level has ceased to a stable or normal range of increases/decreases (e.g., for a predefined period of time).

The overlay notification removal event may be detected based on an indication that a predefined period of time has elapsed since the user has received insulin or entered bolus insulin into a logbook (e.g., in an application). The data indicating the insulin injections may be received from an insulin pump (e.g., insulin pump 116, 118, as shown in FIGS. 1A-1C), a pen (e.g., pen device 103, as shown in FIGS. 1A-1C), and/or another device. The overlay notification removal event may be detected based on an indication that a period of time has elapsed since additional, or alternative, user input has been entered into a logbook or other conditions have been logged in the logbook (e.g., in an application).

The diabetic overlay notification may be removed in response to one or more other indications or messages received from one or more devices (e.g., such as one or more of the remote computing devices 122, the RF beacon device 126, the BGM 106, insulin pumps 116, 118, and/or one or more other devices, as shown in FIGS. 1A-1C). Based on the message type and/or information in the message, the diabetic overlay notification may be removed.

The overlay notification removal event detected for removal of the diabetic overlay notification may be a manual triggering event. For example, the overlay notification removal event may include a user input in the form of an actuation of a button on the computing device or receipt of input on the display of the computing device.

The diabetic overlay notification may be updated dynamically and/or continuously, as described herein, based on updated data related to the user's diabetic condition to provide continuous updating to reduce a number of notifications sent or displayed to a user, while also making information readily accessible and available for monitoring and/or treating a diabetic condition.

The user may unlock the lock screen and/or display the diabetes-related application residing locally on the computing device in response to a selection of the diabetic overlay notification. The local application (e.g., diabetes-related application) may be brought to the foreground on the display of the computing device to allow for the user to access additional features and/or information to enable treatment of the user's diabetic condition. The local application being brought to the foreground on the display of the computing device may also, or alternatively, allow the user to interact with other devices (e.g., paired devices) through the computing device. This may allow the user to adjust parameters for controlling other devices through the local application based on the information on the diabetic overlay notification. For example, the user may access the local application and select a button on the computing device to provide medication to the user (e.g., via one or more messages transmitted from the computing device) and/or adjust thresholds for enabling treatment of the diabetic condition of the user. The overlay notification being updated in response to updated data may allow the user to learn the cause-and-effect relationship between certain events (e.g., receipt of carbs or insulin, performance of exercise, and/or other factors) and the user's medical condition (e.g., diabetic condition and/or blood glucose level).

The computing device may display multiple different types of information in a single overlay notification that may be dynamically updated as information changes. For example, an overlay notification may be provided as a CUE-ME notification that may be dynamically updated based on data being monitored at the computing device and/or one or more other devices (e.g., the insulin pump 116, 118, the CGM 102, the BGM 106, the FGM 111, the pen device 103, etc.). In one example, the computing device may receive different types of information and/or information related to different overlay notification triggering events and group information that is determined to be related and/or associated in memory for being provided in a single overlay notification. Information may be grouped that is received from the same locally executed application and/or determined to relate to the same medical condition of the user (e.g., diabetic condition of the user). Given the importance of data related to the medical condition and/or health of the user (e.g., for treating the condition of the user), it may be beneficial to provide the information, or certain subsets of the information, in a single dynamically updated overlay notification. Additionally, given the limited amount of space on the display of the computing device being operated by the user, it may be beneficial to provide the information, or certain subsets of the information, in a single dynamically updated overlay notification to provide more information and/or allow for the display of other information on the lock screen of the computing device.

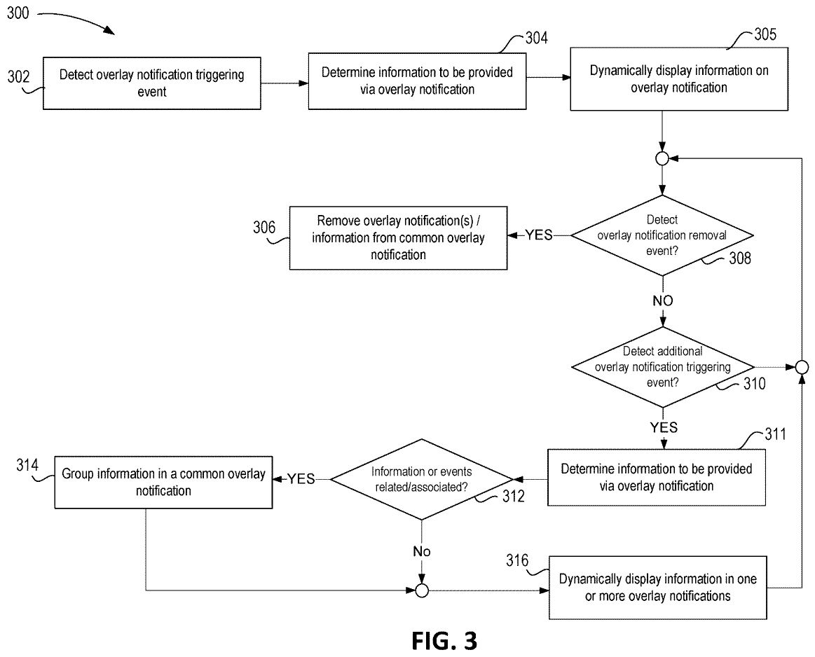

FIG. 3 shows a flowchart of an example process 300 for grouping and displaying information in a common overlay notification. One or more portions of the process 300 may be performed by one or more computing devices. For example, the one or more portions of the process 300 may be performed by one or more mobile devices, such as mobile device 104 shown in FIGS. 1A-1C, and/or one or more remote computing devices, such as remote computing device 122 shown in FIGS. 1A-1C. One or more portions of the process 300 may be stored in memory as computer-readable or machine-readable instructions that may be executed by a processor of the one or more computing devices. Though portions of the process 300 may be described herein as being performed by a particular computing device, the process 300 may be performed by another computing device or distributed across multiple computing devices, such as one or more mobile devices, remote computing devices, and/or one or more other devices.

As shown in FIG. 3, the computing device may detect an overlay notification triggering event at 302. The overlay notification triggering event may be detected based on data received from one or more devices. For example, the overlay notification triggering event may be detected based on data related to the diabetic condition of the user, or other medical or health-related information, as described herein.

After detecting an overlay notification triggering event at 302, the computing device may determine the information to be provided on the overlay notification at 304. The information to be provided on the overlay notification may be related to the diabetic condition of the user. The information to be provided on the overlay notification may be related to another medical condition of the user and/or include other health-related information to be provided to the user, as described herein. The computing device may display the information on the lock screen in an overlay notification to the user at 305. The information may be dynamically displayed and/or updated based on changes in the information over time. For example, the overlay notification may be a CUE-ME notification may include a dynamically updated notification based on updated information received from one or more devices (e.g., the mobile device 104, the insulin pump 116, 118, the CGM 102, the BGM 106, the FGM 111, the pen device 103, etc., as shown in FIGS. 1A-IC). The data may include medical data or data related to the health of the user. For example, the overlay notification may be a diabetic overlay notification that includes a CUE-ME notification that is dynamically updated with the data indicating a current blood-glucose level of the user and/or other information related to a diabetic condition of the user.

The computing device may determine whether an overlay notification removal event is detected at 308. If an overlay notification removal event is detected at 308, the computing device may remove the overlay notification at 306, as described herein.

Additional overlay notification triggering events may be detected after, or within a timeframe of, the detection of the overlay notification triggering event at 302. The additional overlay notification triggering events may be detected prior to or after the information is displayed at 305. For example, the additional overlay notification triggering event may be detected at 310 while the information is being dynamically displayed on the overlay notification in response to the previously detected overlay notification triggering event at 302. In another example, the additional overlay notification triggering event may be detected at 310 prior to the information being displayed at 305 in response to the detection of the overlay notification triggering event at 302. The additional overlay notification triggering event may be detected at 310 within a period of time from the detection of the overlay notification triggering event at 302. For example, each of the overlay notification triggering events may be detected while the application is executing in the background and the computing device is in the sleep state.

If an additional overlay notification triggering event is detected at 310, the computing device may determine the information to be provided on the overlay notification at 311 based on the additional overlay notification trigger detected at 310. The information may be the same information or different information than the information determined to be provided at 304 in response to the detection of the overlay notification triggering event at 302. The computing device may group different types of information in the same overlay notifications or provide different overlay notifications for providing the different types of information to the user.

The computing device may determine, at 312, whether the information determined at 311 based on the overlay notification triggering event detected at 310 is related to and/or associated with the information determined at 304 based on the overlay notification triggering event detected at 302. Additionally, or alternatively, the computing device may determine, at 312, whether the overlay notification triggering event detected at 302 is related to and/or associated with the overlay notification triggering event detected at 310. If the overlay notification triggering events are related and/or associated, the information to be provided based on the events may be related and/or associated. Related and/or associated information may be grouped in a common overlay notification at 314. This may allow related and/or associated information that is to be displayed within the same time frame to be grouped in a common overlay notification.

The information and/or triggering events provided by the same application (e.g., having a particular application identifier) may be related and/or associated in memory for grouping the information in a common overlay notification. For example, information and/or triggering events provided by the same medical (e.g., diabetes) or health-related application may be related and/or associated in memory.

The information and/or triggering events provided by the same device (e.g., having a particular device identifier) may be related and/or associated in memory for grouping the information in a common overlay notification. For example, information and/or triggering events provided by the mobile device 104 the insulin pump 116, 118, the CGM 102, the BGM 106, the FGM 111, the pen device 103, and/or the remote computing device 122 may each be associated with a separate device identifier that may be used for grouping information in a common overlay notification.

The information and/or triggering events related to the same medical condition of the user may be related and/or associated in memory for grouping the information in a common overlay notification. For example, information and/or triggering events provided for treatment of a diabetic condition or another common medical condition may be used for grouping information in a common overlay notification.

The information and/or triggering events related to the same health-related category may be related and/or associated in memory for grouping the information in a common overlay notification. A health-related category may include information and/or triggering events related to mealtime events, exercise events, medical conditions, and/or other health-related categories. Each health-related category may be used for grouping information in a common overlay notification. Each health-related category may have its own identifier. Each health-related category may be predefined and/or user-defined.

The information and/or triggering events related to the same health-related sub-category may be related and/or associated in memory for grouping the information in a common overlay notification. For example, a sub-category of mealtime events may include particular meals or snack times indicated by an identifier. A sub-category of exercise events may include particular exercises indicated by an identifier. A sub-category of a particular medical condition may include information related to a particular medication or type of information that may be used to treat the medical condition. In one example, information related to a user's glucose levels may be grouped together in a common overlay notification for treating of a diabetic condition.

The information and/or triggering events related to a medical or health-related condition of the user may be related and/or associated in memory with one or more device identifiers for enabling a grouping of the medical or health-related information with status information related to the one or more devices corresponding to the device identifiers. The status of one or more devices may inform the user as to the accuracy of the medical or health-related information being provided by such devices. The medical or health-related information of the user may be provided by a device that has malfunctioned. For example, the medical or health-related information may include a graph that indicates the glucose level of a user over time. At a certain period of time, the glucose level of the user may increase/decrease and appear inaccurate to the user. The status of a CGM, FGM, BGM, or other sensor device may be provided in a common overlay notification as the information related to the glucose level of the user to allow the user to determine whether the status of the device is affecting the change in the blood glucose level of the user. The information related to the device may include whether it is on/off, paired/unpaired to the computing device, the age of the device, whether it has been calibrated or the last time it was calibrated, and/or other information related to a state of the device. In one example, a CGM may have different blood glucose monitoring states (e.g., calibrated, uncalibrated, first calibration state, second calibration state, etc.) that allow for more/less accurate blood glucose readings. This information may be provided in the device information that is grouped with the blood glucose level of the user to inform the user as to the accuracy of the blood glucose information being provided.

The computing device may determine whether the information and/or triggering events are related and/or associated by analyzing information from one or more devices and/or the information to be provided on the overlay notification. For example, the computing device may analyze information from one or more devices to detect common identifiers of information and/or triggering events. The computing device may determine that the information and/or triggering events are related when one or more (e.g., a threshold number) of common identifiers are detected. The computing device may also, or alternatively, perform textual analysis to match terms and/or identifiers in data. For example, if the computing device identifies predefined terms and/or a threshold number of similar terms in the information to be provided via the overlay notification and/or the triggering event from which the information is to be provided, then the information may be grouped in a common overlay notification.

If the information and/or triggering events are determined to be related and/or associated at 312, the information to be provided on the lock screen may be grouped into a common overlay notification at 314. The computing device may group the information to avoid displaying multiple separate overlay notifications. The information may be dynamically displayed and independently updated in the common diabetic overlay notification at 316. If the information and/or triggering events are determined to be unrelated and/or disassociated at 312, the information to be provided on the graphical user interface may be provided in separate overlay notifications at 316.

The computing device may continue to detect overlay notification removal events at 308 for removing the overlay notification and/or information from a common overlay notification at 306. An overlay notification removal event may be detected that corresponds to a particular type of information being displayed and that information may be removed from the common overlay notification. As an overlay notification removal event may correspond to one or more types of information being displayed in a common overlay notification, one or more types of information may be removed from the common overlay notification in response to detecting an overlay notification removal event at 308.

The computing device may continue to detect overlay notification triggering events at 310 for determining information at 311 that may be added to a common overlay notification or displayed in a separate overlay notification at 316. In one example, the computing device may detect an overlay notification triggering event when the user's current blood glucose level indicates that the user has entered a hyperglycemic state or is within a predefined threshold of entering the hyperglycemic state. In response to this overlay notification triggering event, the computing device may display a diabetic overlay notification as a CUE-ME notification including information indicating the user's hyperglycemic blood glucose level and information indicating the user's current blood glucose level. The user may then receive a delivery of bolus insulin or carbohydrates. In response to the bolus insulin delivery or carbohydrates, one or more devices (e.g., delivery device, one or more sensor devices, and/or one or more entries into a logbook) may indicate to the computing device that the user has received the bolus insulin or carbohydrates. The computing device may determine in response to the indication of the bolus insulin delivery or delivery of carbohydrates information indicating the user's current glucose level over a predefined period of time (e.g., three hours) under the impact of insulin or after the delivery of carbohydrates. In response to the additional overlay notification triggering event (e.g., the indication of the delivery of bolus insulin to the user), the computing device may display the common overlay notification with information that indicates: (1) the user's current blood glucose level, (2) the user's hyperglycemic blood glucose level, and (3) the user's current glucose level over the next three hours under the impact of the insulin.

A common overlay notification may include information from a predefined number of overlay notification triggering events, a predefined number of different types of information generated from overlay notification triggering events, or a predefined number of groupings. As a common overlay notification may be of a predefined size, the different types of information that may be grouped in a common overlay notification may depend on the amount/size of information being provided and the size of the overlay notification. If a threshold is reached for grouping information into a common overlay notification, the computing device may display information in another overlay notification at 316.

The computing device may display the common diabetic overlay notification for a predefined period of time before removing the common diabetic overlay notification (e.g., if the computing device doesn't detect another overlay notification removal event or an interaction with the overlay notification or another portion of the computing device).

When providing information in an overlay notification that is displayed as a lock screen notification, which may be presented as a push notification on a lock screen, sensitive information may be displayed on the computing device. As medical and/or health-related information may be sensitive information that may be displayed in a lock screen notification on the lock screen prior to the user providing input, such as a security key and/or provide biometric data (e.g., fingerprint, facial features for facial recognition, and/or other biometric data), to authenticate the user, the lock screen notification may be provided in a manner to protect the user data, as described herein.

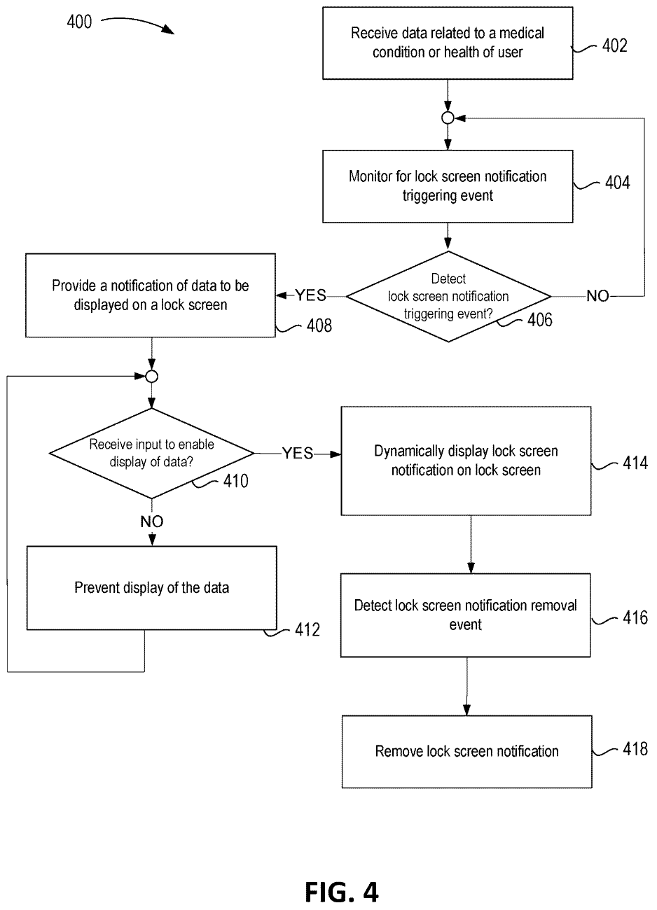

FIG. 4 shows a flowchart of an example process 400 for protecting user data when displaying on lock screen notification. One or more portions of the process 400 may be performed by one or more computing devices. For example, the one or more portions of the process 400 may be performed by one or more mobile devices, such as mobile device 104 shown in FIGS. 1A-1C, and/or one or more remote computing devices, such as remote computing device 122 shown in FIGS. 1A-1C. One or more portions of the process 400 may be stored in memory as computer-readable or machine-readable instructions that may be executed by a processor of the one or more computing devices. Though portions of the process 400 may be described herein as being performed by a particular computing device, the process 400 may be performed by another computing device or distributed across multiple computing devices, such as one or more mobile devices, remote computing devices, and/or one or more other devices.

As shown in FIG. 4, the computing device may receive data related to a medical condition (e.g., diabetic condition) or health of a user at 402. The received data may come from one or more devices (e.g., the mobile device 104, the insulin pump 116, 118, the CGM 102, the BGM 106, the FGM 111, the pen device 103, etc., as shown in FIGS. 1A-1C). The received data may include information related to a chronic condition, medical information, and/or other health-related information that may be sensitive to the user. For example, the chronic condition, medical information, and/or health-related information may comprise information related to the diabetic condition of the user, such as information indicating abnormal glucose levels, high blood pressure, abnormal heart beats per minute (bpm), a current blood-glucose level of a user, dosing information for medication (e.g., insulin), the user's hyperglycemic blood glucose level, and/or other information related to a chronic condition, medical information, and/or other health-related information.

After receiving data related to a medical condition (e.g., diabetic condition) or health of a user at 402, the computing device may monitor for a lock screen notification triggering event at 404. If the computing device fails to detect the lock screen notification triggering event at 406, the computing device may continue to monitor for a lock screen notification triggering event based on data that continues to be received.

After the computing device detects the lock screen notification triggering event at 406, the computing device may provide a notification, at 408, that data is ready to be displayed on the lock screen of the computing device. The computing device may prevent the display of the data at 412 until input is received at 410 from the user to enable display of the data in the form of the lock screen notification at 414. The lock screen notification may include an obscured display of the data on the lock screen. For example, the computing device may blur, redact, or otherwise prevent a readable display of the data in the lock screen notification and/or predefined portions of the lock screen notification that include sensitive information (e.g., medical information and/or information related to the health of the user).

The notification that is provided at 408 may include an indication that the data may be sensitive and/or related to the particular medical condition (e.g., diabetic condition) or health of the user. The notification may include an indication that data is ready to be displayed in the lock screen notification. The notification may be provided as text and/or an image displayed on the computing device. The notification may be displayed in relation to the lock screen notification (e.g., above, below, beside, or overlaid on top of). The notification may be provided as a link, a button, or other format capable of receiving input by the user. In response to the user input on the notification, the user may provide input to enable the display of the data in the lock screen notification. For example, in response to the input from the user, the computing device may provide an additional screen to allow the user to input a security key, biometric data, and/or provide other input to allow the data to be displayed in the lock screen notification. The input for enabling the display of the data in the lock screen notification may be the same as or different from the input for unlocking the lock screen on the computing device.

After the computing device dynamically displays the lock screen notification at 414 on the lock screen of the computing device, the lock screen notification may be dynamically and/or continuously updated based on updated data, as described herein. The computing device may detect a lock screen notification removal event at 416. After detecting the lock screen notification removal event at 416, the computing device may remove the lock screen notification from the lock screen of the computing device at 418.

Though the process 400 may be described as providing the notification of data to be displayed in response to detecting a lock screen notification triggering event from the monitored data, the computing device may provide the notification in response to a user input. For example, the computing device may display a button or link persistently or in response to a lock screen notification triggering event on the lock screen of the computing device to solicit input from the user. The button or link may enable the option for a lock screen to be provided by the user on demand to access the data, without the user accessing the local application on the computing device. For example, a diabetic lock screen notification may be activated by the user in the event the user wants a view of a glucose state or trend in a blood glucose level at a specific moment. The button or link may be actuated by the user and/or held down to display the lock screen notification (e.g., for a set duration or until the user ceases the actuation or provides another actuation). The lock screen notification may be visible while the button is pressed for a quick, clandestine, view of the data, without having to access the app. While the lock screen notification is being displayed, the lock screen notification may be dynamically and/or continuously updated. The settings associated with the local application (e.g., diabetes-related application) may provide an option that may be selected by the user for the privacy settings to protect the data.

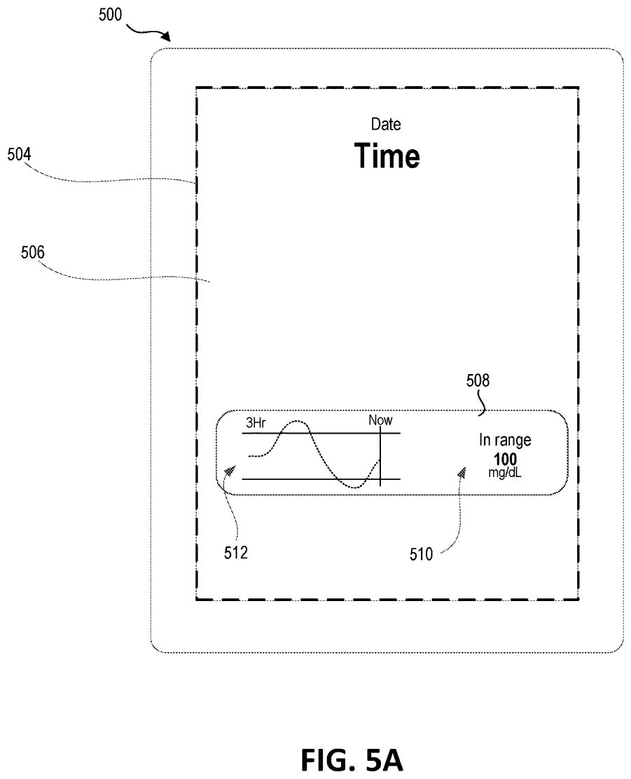

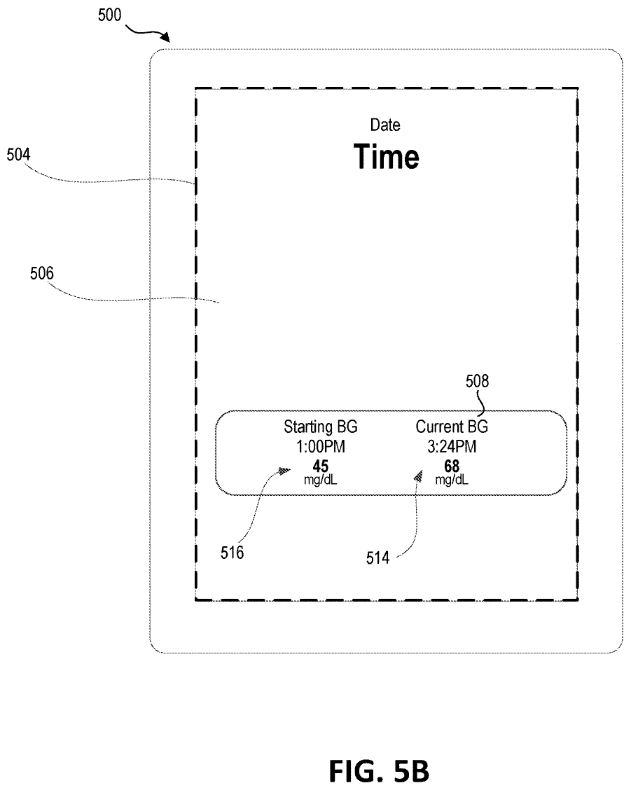

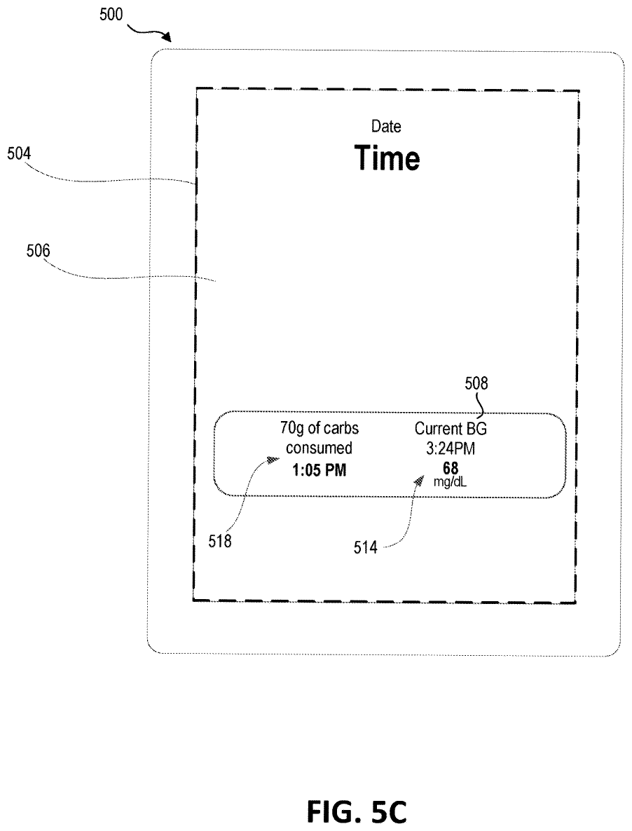

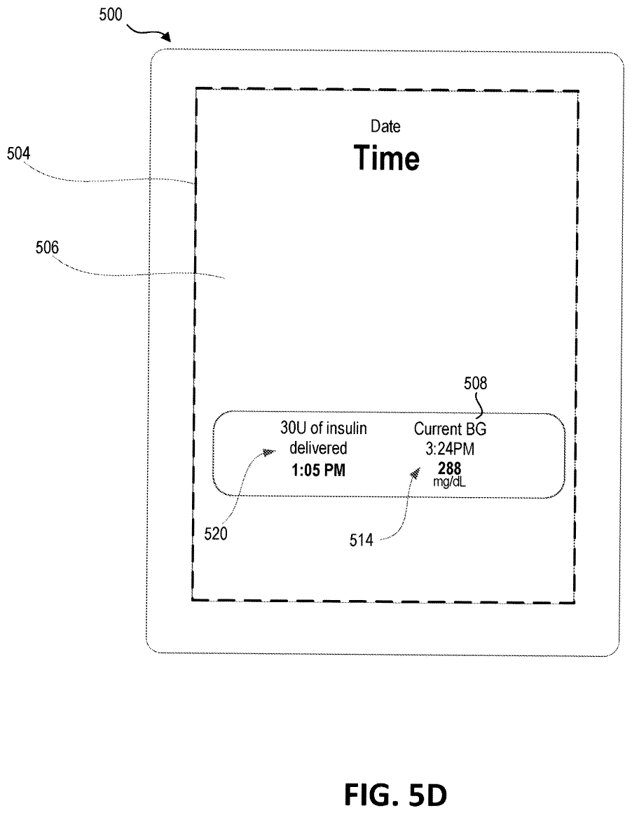

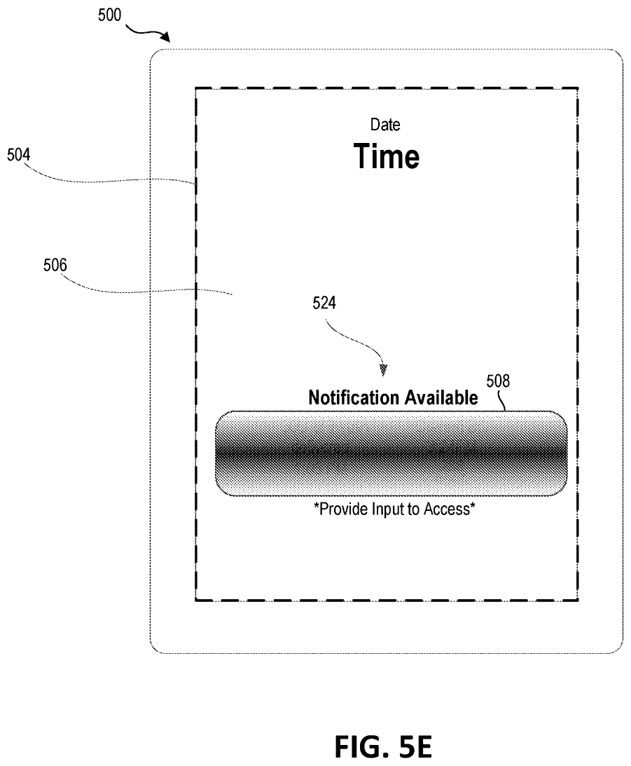

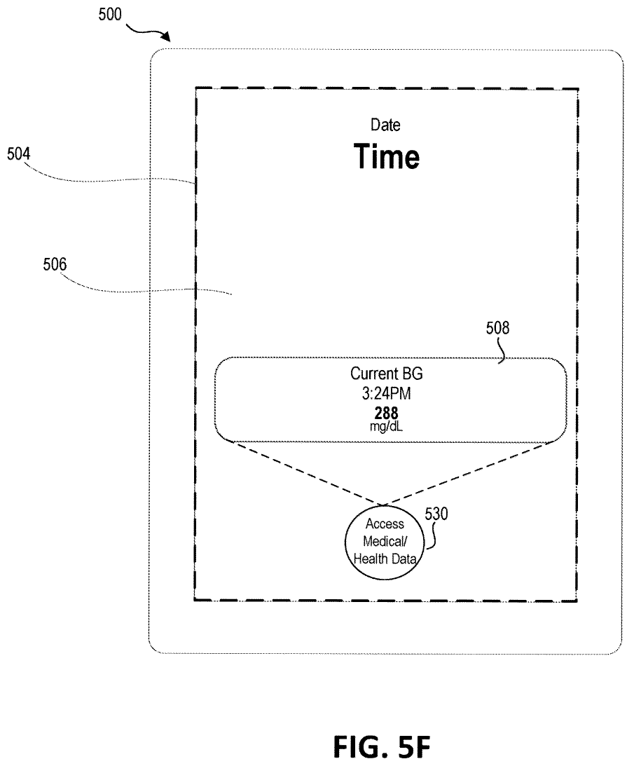

FIGS. 5A-5F depict example GUIs illustrating overlay notifications on a display 504 of a computing device 500. The GUIs are generated by software or firmware on the computing device 500. As shown in FIG. 5A, an overlay notification 508 may be displayed on the display 504 of the computing device 500. The overlay notification 508 may be overlaid on the graphical user interface 506 (e.g., lock screen, home screen, call screen, and/or other graphical user interface), when the graphical user interface 506 is displayed to the user. The overlay notification 508 may be provided as a diabetic overlay notification providing information related to the diabetic condition of the user. The information provided on the diabetic overlay notification may be provided from information entered into a logbook, sensor information, or information from one or more other devices (e.g., the mobile device 104, the insulin pump 116, 118, the CGM 102, the BGM 106, the FGM 111, the pen device 103, the remote computing device 122, and/or another computing device). Though the overlay notification 508 may include information related to a diabetic condition of the user, the overlay notification may provide other information related to a medical condition or health of the user.

As shown in FIG. 5A, the overlay notification 508 may include one or more different types of information. For example, the overlay notification 508 may include an indication 510 that the user's blood glucose level is within a predefined range and/or identify the predefined blood glucose range for the user. The overlay notification 508 may include an indication 512 of the blood glucose level of the user over a period of time. For example, the overlay notification 508 may include a graphical indication that illustrates the current blood glucose level of the user and the blood glucose level of the user over a period of time. The graph may show the blood glucose level of the user relative to the predefined range for the user. The predefined range may indicate a blood glucose level of the user when the user may enter an extreme diabetic state or a predicted extreme diabetic state (e.g., hypoglycemia) associated with the user, or an indication to the user to consume carbohydrates or insulin to help self-treat the extreme diabetic state. The overlay notification 508 may include grouped information into a single overlay notification, as described herein.

As shown in FIGS. 5B-5D, the overlay notification 508 may include other types of information related to the diabetic condition of the user. For example, the overlay notification 508 may include an indication 516 of the user's starting blood glucose level. The starting blood glucose level may be displayed with a time at which the starting blood glucose level was measured. The indication 516 of the starting blood glucose level of the user may be displayed with the indication 514 of the current blood glucose level of the user on the overlay notification 508.

As shown in FIG. 5C, the overlay notification 508 may include an indication 518 that carbohydrates have been consumed by the user. The indication 518 may identify a number of carbohydrates consumed and/or a time at which the carbohydrates were consumed. The indication 518 may be provided in the overlay notification with the indication 514 of the current blood glucose level of the user.

As shown in FIG. 5D, the overlay notification 508 may include an indication 520 that insulin has been delivered to the user. The indication 520 may identify an amount of insulin delivered and/or a time at which the insulin was delivered. The indication 520 may be provided in the overlay notification 508 with the indication 514 of the current blood glucose level of the user.

As shown in FIG. 5E, the overlay notification 508 may be provided in a manner to prevent the display of certain data to the user until input is received from the user to enable the display of the data. For example, the overlay notification 508 may include an obscured display of the data on the lock screen, such as a blurred, redacted, or otherwise obscured display of the data to prevent a readable display of the data in the overlay notification and/or predefined portions of the overlay notification that include sensitive information (e.g., medical information and/or information related to the health of the user). The overlay notification 508 may be provided with a notification 524 that includes an indication that the information in the overlay notification 508 is available and ready to be displayed. The notification 524 may indicate that the data in the overlay notification 508 may be sensitive and/or related to the particular medical condition (e.g., diabetic condition) or health of the user. The notification 524 may provide instructions to the user to allow the user to access the data in the overlay notification 508. For example, the notification 524 and/or the overlay notification 508 may be provided in a format capable of receiving input by the user. The user may select the notification 524 and/or the overlay notification 508 to enable the user to provide an input (e.g., security code, biometric data, or other input) to cause the overlay notification 524 to display the data. In response to the user input on the notification 524 and/or the overlay notification 508, the computing device 500 may provide an additional screen to allow the user to input a security key, biometric data, and/or provide other input to allow the data to be displayed in the overlay notification. The input provided by the user may enable the display of the data in the overlay notification.

As shown in FIG. 5F, the overlay notification 508 may be provided in response to a user actuation of a button 530 displayed on the graphical user interface 506 (e.g., lock screen, home screen, call screen, and/or other graphical user interfaces). For example, the computing device 500 may display a button 530 persistently on the graphical user interface 506 of the computing device 500 to solicit input from the user. The button 530 may enable the option for the overlay notification 508 to be provided by the user on demand to access the data on the overlay notification 508, without the user accessing the local application on the computing device 500. The overlay notification 508 may be visible while the button 530 is pressed, or the overlay notification 508 may be provided in a manner to prevent the data from being displayed until further input is received from the user (e.g., as shown in FIG. 5E).

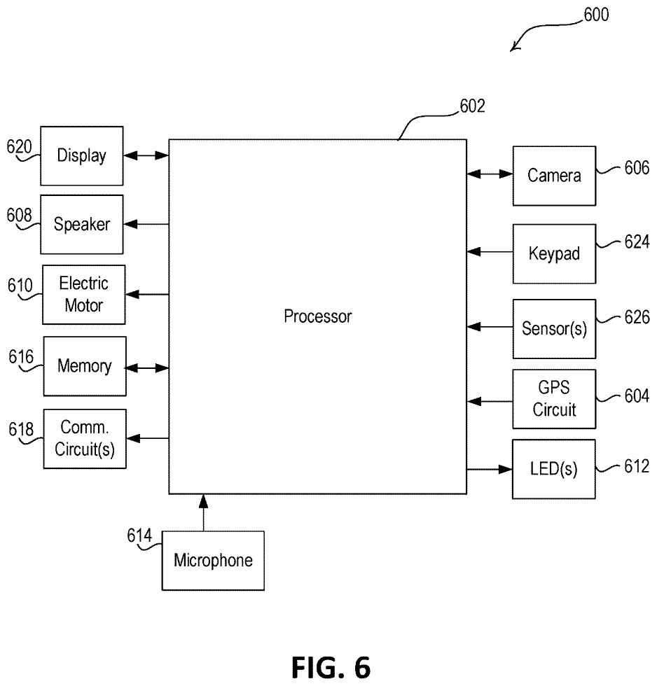

FIG. 6 is a block diagram of an example computing device 600. The computing device 600 may be a mobile computing device, such as a tablet, a cellular phone, a wearable device, a CGM controller device, a remote computing device, or another computing device, for example. As shown in FIG. 6, the computing device 600 may include a processor 602 for controlling the functionality of the computing device 600. The processor 602 may include one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 602 may perform signal coding, data processing, power control, image processing, input/output processing, or any other functionality that enables the computing device 800 to perform as described herein.

The processor 602 may store information in or retrieves information from the memory 616. The memory 616 includes a non-removable memory or a removable memory. The non-removable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The processor 602 may access the memory 616 for executable instructions or other information that is used by the computing device 600. For example, the memory 616 may include computer-readable or machine-readable instructions that may be executed by the processor 602 for performing one or more methods, processes, or procedures, or portions thereof, as described herein.

The computing device 600 may include a camera 606 that is in communication with the processor 602. The camera 606 may be a digital camera or other optical device capable of generating images or videos (e.g., image sequences) for being captured at the computing device 600. The camera 606 may include a lighting device capable of flashing to in response to signals from the processor 602.

The computing device 600 may include one or more communication circuits 618. The processor 602 may be in electrical communication with the communication circuit 618 for sending or receiving information. The communication circuit 618 may be capable of performing wired and/or wireless communications. For example, the communication circuit 618 may include one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. In some embodiments, one or more communication circuits 618 are capable of performing infrared (IR) communications.

The processor 602 may be in electrical communication with a keypad 624 for providing input to the processor 602. The keypad 624 may include one or more keys for receiving input from a user. The keypad 624 may include hard or soft keys for which the function of the keys changes as a user performs selections.

Other input into the processor 602 may be provided by one or more sensors 626. The sensors 626 include a motion sensor, a proximity sensor, a heartrate monitoring sensor, an accelerometer, a gyroscope, and/or another sensor on the computing device 600. The motion sensor may transmit infrared signals or use image processing to sense movement. The proximity sensor may transmit infrared signals to detect when an object is within a predefined proximity. The heartrate monitoring sensor may implement photoplethysmography to detect the amount of blood flow in the user. The heartrate monitoring sensor may include one or more LED or photodiodes to detect the amount of blood flow in the user. The heartrate monitoring sensor may implement infrared technology to detect the amount of blood flow in the user. The heartrate monitoring sensor may take an electrocardiogram (ECG) and detect information about the user's heartrate from the ECG. The accelerometer may measure the non-gravitational acceleration of the computing device 800 in a given direction. The accelerometer may respond to vibrations associated with movement in a given direction. The measurements from the accelerometer may be used by the processor 602 to determine the magnitude or direction of the relative movement of the computing device 600, or the user's relative position (e.g., standing, sitting, or lying down). The gyroscope may be used to determine the orientation of the computing device 600.

The processor 602 may be in electrical communication with or generate images on a display 620 for providing information to a user. The communication between the display 620 and the processor 602 may be a two-way communication, as the display 620 may include a touch screen module capable of receiving information from a user and providing such information to the processor 602. For example, the display 620 may provide soft buttons for selection by a user that are recognized by the touch screen module and provided to the processor 602 as input.

The processor 602 may be in electrical communication with or control a speaker 608. The speaker 608 may provide an audible sound (e.g., tone, beep, or buzz) in response to a triggering event detected by the processor 602.

The computing device 600 may include an electric motor 610 that is in electrical communication with or controlled by the processor 602. The electric motor 610 may rotate and causes the computing device 600 to vibrate (e.g., to indicate an alert) in response to a triggering event detected by the processor 602.

The processor 602 may be in electrical communication with or receive information from a microphone 614. For example, the processor 602 may receive audio signals via the microphone 614.

The computing device 600 may include a global positioning system (GPS) circuit 604. The GPS circuit 604 may be capable of receiving GPS information. The processor 602 may be capable of determining the GPS coordinates (e.g., latitude and longitude) of the computing device 600 based on the GPS information received via the GPS circuit.

The computing device 600 may include a visual indicator, such as one or more light-emitting diodes (LEDs) 612. In some embodiments, one or more LEDs 612 are illuminated or flashed to provide an alert or communicate other information to the user (e.g., low battery or turning on of the device).

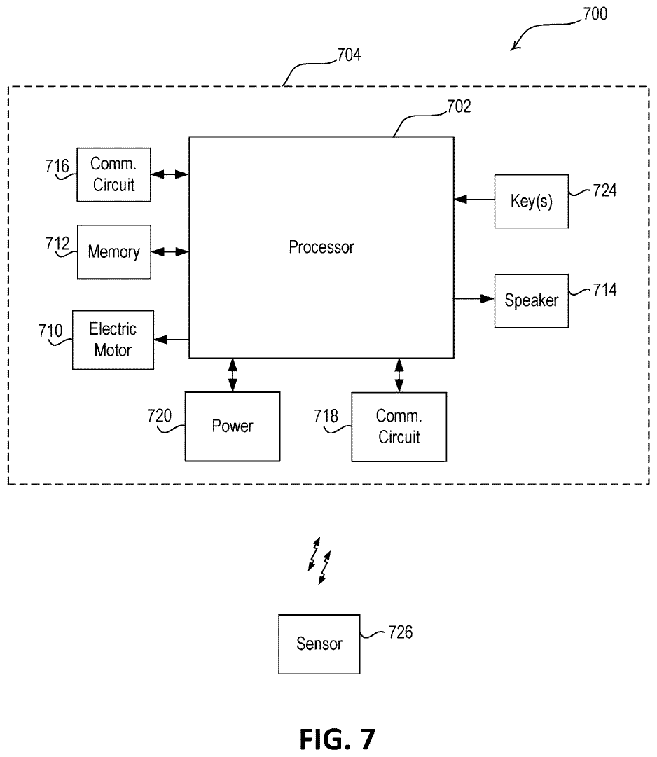

FIG. 7 is a block diagram of an example blood glucose monitoring device 700. In some embodiments, the blood glucose monitoring device 700 is a CGM or FGM, for example. The blood glucose monitoring device 700 may include a subcutaneous sensor 726 that is used to sense and monitor the amount of glucose in interstitial fluid of the user. Data is transmitted from the sensor 726 to a transmitting device 704. When the blood glucose monitoring device 700 is a CGM, the transmitting device 704 is located directly over the sensor 726 and wirelessly powers the data transfer from the sensor 726 via power supply 720. When the blood glucose monitoring device 700 is an FGM, the transmitting device 704 is a mobile device or other reader device that instantaneously receives the blood glucose information from the sensor 726 when the device is within the RF range of the sensor 726.

The transmitting device 704 receives data communications from the sensor 726 via a communication circuit 718. The communication circuit 718 may be in electrical communication with a processor 702. The processor 702 may include one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 702 may perform signal coding, data processing, power control, input/output processing, or any other functionality that enables the transmitting device 704 to perform as described herein.

The transmitting device 704 may include another communication circuit 716 for communicating with other devices. The processor 702 may be in electrical communication with the communication circuit 716 for sending or receiving information. The communication circuits 716, 718 are capable of performing wired or wireless communications. For example, the communication circuits 716, 718 may include one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. The communication circuits 716, 718 may communicate using the same RF protocol or a different RF protocol.

The processor 702 may store information in or retrieves information from the memory 712. The memory 712 may include a non-removable memory or a removable memory. The non-removable memory may include random-access memory (RAM), read-only memory (ROM), a hard disk, or any other type of non-removable memory storage. The removable memory may include a subscriber identity module (SIM) card, a memory stick, a memory card (e.g., a digital camera memory card), or any other type of removable memory. For example, the memory 712 may include computer-readable or machine-readable instructions that may be executed by the processor 702 for performing one or more methods, processes, or procedures, or portions thereof, as described herein. The processor 702 may access the memory 712 for executable instructions or other information that is used by the transmitting device 704. The processor 702 is in electrical communication with one or more input keys 724 for providing input to the processor 702.

The processor 702 may be in electrical communication with or control a speaker 714. The speaker 714 may provide an audible sound (e.g., tone, beep, or buzz) in response to a triggering event detected by the processor 702.

The blood glucose monitoring device 700 may include an electric motor 710 that is in electrical communication with or controlled by the processor 702. The electric motor 710 may rotate and causes the blood glucose monitoring device 700 to vibrate (e.g., to indicate an alert) in response to a triggering event detected by the processor 702. The electric motor 710 may provide an alert to supplement the audible alarm or replace the audible alarm provided by the speaker 714.

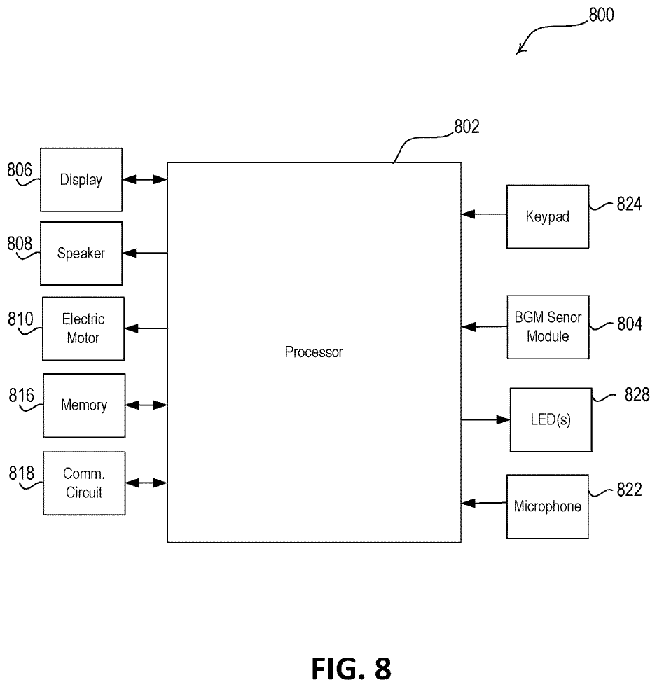

FIG. 8 is a block diagram of an example blood glucose meter (BGM) device 800. As shown in FIG. 8, the BGM device 800 may include a processor 802 for controlling the functionality of the BGM device 800. The processor 802 may include one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 802 may perform signal coding, data processing, power control, image processing, input/output processing, and/or any other functionality that enables the BGM device 800 to perform as described herein.

The processor 802 may store information in or retrieve information from the memory 816. The memory 816 may include a non-removable memory or a removable memory. The non-removable memory may include random-access memory (RAM), read-only memory (ROM), a hard disk, and/or any other type of non-removable memory storage. The removable memory may include a subscriber identity module (SIM) card, a memory stick, a memory card (e.g., a digital camera memory card), and/or any other type of removable memory. The memory 816 may include computer-readable or machine-readable instructions that may be executed by the processor 802 for performing one or more methods, processes, or procedures, or portions thereof, as described herein. The processor 802 may access the memory 816 for executable instructions or other information that is used by the BGM device 800.

The BGM device 800 may include one or more communication circuits 818. The processor 802 may be in electrical communication with the communication circuit 818 for sending or receiving information. The communication circuit 818 may be capable of performing wired and/or wireless communications. For example, the communication circuit 818 may include one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. In some embodiments, one or more communication circuits 818 may be capable of performing infrared (IR) communications.

The processor 802 may be in electrical communication with a keypad 824 for providing input to the processor 802. The keypad 824 may include one or more keys for receiving input from a user. The keypad 824 may include hard or soft keys for which the function of the keys changes as a user performs selections.

Other input into the processor 802 may be provided by the BGM sensor module 804. The BGM sensor module 804 may include a blood glucose measuring engine that may analyze blood samples provided by a patient on a blood glucose measurement strip and measure the amount of blood glucose in the samples.

The processor 802 may be in electrical communication with or generate images on a display 806 for providing information to a user. The communication between the display 806 and the processor 802 may be a two-way communication, as the display 806 may include a touch screen module capable of receiving information from a user and providing such information to the processor 802. For example, the display 806 may provide soft buttons for selection by a user that are recognized by the touch screen module and provided to the processor 802 as input.

The processor 802 may be in electrical communication with or control a speaker 808. The speaker 808 may provide an audible sound (e.g., tone, beep, or buzz) in response to a triggering event detected by the processor 802.

The BGM device 800 may include an electric motor 810 that is in electrical communication with or controlled by the processor 802. The electric motor 810 may rotate and cause the BGM device 800 to vibrate (e.g., to indicate an alert) in response to a triggering event detected by the processor 802. The electric motor 810 may provide an alert to supplement the audible alarm or replace the audible alarm provided by the speaker 808.

The processor 802 may be in electrical communication with or receive information from a microphone 822. For example, the processor 802 may receive audio signals via the microphone 822.

The BGM device 800 may include a visual indicator, such as one or more one or more light-emitting diodes (LEDs) 828. In some embodiments, one or more LEDs 828 are illuminated or flashed to provide an alert or communicate other information to the user (e.g., low battery or turning on of the device).

Although features, elements, and functions are described above in particular combinations, a feature, element, or function is used alone or in any combination with the other features, elements, or functions. Various presently unforeseen or unanticipated alternatives, modifications, variations, or improvements may be subsequently made that are also intended to be encompassed by the following claims.

The methods described herein are implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), removable disks, and optical media such as CD-ROM disks, and digital versatile disks (DVDs).

### ENUMERATED EMBODIMENTS

The following list of enumerated embodiments further described herein.
EE 1. A method comprising:
receiving data related to a diabetic condition of a person;
detecting, based on the received data, an overlay notification triggering event associated with the diabetic condition;
in response to detecting the overlay notification triggering event, dynamically displaying a diabetic overlay notification on a graphical user interface of the computing device, wherein the diabetic overlay notification is associated with a diabetes application, wherein the overlay notification is displayed for a period of time or until an overlay notification removal event associated with the diabetic condition is detected, wherein the period of time is configured to be a predefined period of time from the overlay notification triggering event associated with the diabetic condition;
detecting, based on the received data related to the diabetic condition, the overlay notification removal event; and
in response to detecting the overlay notification removal event, removing the diabetic overlay notification from the graphical user interface of the computing device.
EE 2. The method of EE 1, wherein the received data is data from a continuous glucose monitor.
EE 3. The method of EE 2, wherein the received data is related to a chronic condition of the user for being monitored or device malfunctions.
EE 4. The method of EE 2, wherein the overlay notification triggering event is detected when the received data from the continuous glucose monitor indicates a glucose level above an upper threshold or below a lower threshold, wherein the overlay notification removal event is detected when the received data from the continuous glucose monitor indicates a glucose level within the predefined range.
EE 5. The method of EE 1, wherein the overlay notification triggering event is manually or automatically configured.
EE 6. The method of EE 1, wherein the overlay notification triggering event is configured to be manually turned on or turned off.
EE 7. The method of EE 1, wherein the overlay notification triggering event is detected based on one of an indication of an extreme diabetic state, an indication of an abnormal increase or decrease of blood glucose level within a predefined period of time, an indication of a performance of exercise by the person, an indication of mealtime, or an indication that the person received a predefined amount of short-acting insulin within a predefined period of time.
EE 8. The method of EE 1, wherein the method further comprises:
determining that the computing device is in a sleep state;
receiving a wake indicator before displaying the overlay notification; and
waking the computing device from the sleep state and displaying the overlay notification in response to the overlay notification triggering event.
EE 9. The method of EE 1, wherein the overlay notification removal event is manually or automatically configured.
EE 10. The method of EE 9, wherein the overlay notification removal event is configured to be manually turned on or turned off.
EE 11. The method of EE 1, wherein the overlay notification removal event is detected based on one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.
EE 12. The method of EE 1, wherein the graphical user interface is one of a lock screen, a home screen, or a call screen displayed on the computing device.
EE 13. The method of EE 12, wherein the graphical user interface is the lock screen, the method further comprising:
unlocking the screen and displaying a diabetes application in response to a selection of the overlay notification.
EE 14. A method comprising:
receiving data related to a diabetic condition of a person;
detecting, based on the received data, a first overlay notification triggering event associated with the diabetic condition;
determining, based on the first overlay notification triggering event, first information related to a first diabetic overlay notification;
detecting, based on the received data, a second overlay notification triggering event associated with the diabetic condition;
determining, based on the second overlay notification triggering event, second information related to a second diabetic overlay notification;
in response to detecting the second overlay notification triggering event within a first period of time of the first overlay notification triggering event, grouping the first information and the second information in a common diabetic overlay notification;
dynamically displaying the common diabetic overlay notification that includes the grouped first information and second information on a graphical user interface of a computing device, wherein the common diabetic overlay notification is displayed for a second period of time or until an overlay notification removal event associated with the diabetic condition is detected, wherein the second period of time is a predefined period of time from the second overlay notification triggering event;
detecting, based on the received data, an overlay notification removal event associated with at least one of the first overlay notification or the second overlay notification; and
in response to detecting the overlay notification removal event, removing at least one of the first information or the second information from the lock screen of the computing device.
EE 15. The method of EE 14, wherein the received data is data from a continuous glucose monitor.
EE 16. The method of EE 15, wherein the received data is related to a chronic condition than needs to be monitored or device malfunctions.
EE 17. The method of EE 15, wherein at least one of the first overlay notification triggering event or the second overlay notification triggering event is detected when the received data from the continuous glucose monitor indicates a glucose level above an upper threshold or below a lower threshold, wherein the overlay notification removal event is detected when the received data from the continuous glucose monitor indicates a glucose level within the predefined range.
EE 18. The method of EE 14, wherein at least one of the first overlay notification triggering event or the second overlay notification triggering event are one of an indication of an extreme diabetic state, an indication of an abnormal increase or decrease of blood glucose level within a predefined period of time, an indication of exercise performed by the person, a mealtime, or an indication that the person received short-acting insulin within a predefined period of time.
EE 19. The method of EE 14, wherein the overlay notification removal event is one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.
EE 20. The method of EE 14, further comprising:
determining that the first overlay notification and the second overlay notification are related or associated in memory prior to grouping the first information and the second information in the common diabetic overlay notification.
EE 21. The method of EE 14, further comprising:
determining that the first information and the second information are related or associated in memory prior to grouping the first information and the second information in the common diabetic overlay notification.
EE 22. The method of EE 21, wherein the first information and the second information are determined to be related and/or associated in memory by being provided by the same application, received in messages from the same device, related to the same condition, or related to the same health-related category or sub-category.
EE 23. The method of EE 21, further comprising:
analyzing the first information and the second information to detect common identifiers; and
wherein the first information and the second information are determined to be related based on the detection of at least one common identifier.
EE 24. The method of EE 14, wherein the graphical user interface is one of a lock screen, a home screen, or a call screen displayed on the computing device.
EE 25. A method comprising:
receiving data related to a diabetic condition of a person;
detecting, based on the received data, a lock screen notification triggering event associated with the diabetic condition;
in response to detecting the lock screen notification triggering event, providing a notification of data related to the diabetic condition or the person to be displayed on a lock screen of a computing device via a diabetic lock screen notification;
preventing display of the data related to the diabetic condition or the person until an input is received from a user to enable the display of the data;
receiving, via the lock screen, the input from the user to enable the display of the data related to the diabetic condition or the person on the lock screen; and
in response to the receipt of the input from the user, dynamically displaying the lock screen notification on the lock screen of the computing device.
EE 26. The method of EE 25, wherein the received data is data from a continuous glucose monitor.
EE 27. The method of EE 26, wherein the received data from the chronic condition than needs to be monitored or device malfunctions.
EE 28. The method of EE 25, wherein the lock screen notification triggering event is manually configured.
EE 29. The method of EE 25, wherein the lock screen notification triggering event is configured to be manually turned on or turned off.
EE 30. The method of EE 25, wherein the lock screen notification triggering event is configured to be one of an extreme diabetic state, an abnormal increase or decrease of blood glucose level within a predefined period of time, an exercise, a meal, a snack or an indication that the person received short-acting insulin within a predefined period of time.
EE 31. The method of EE 25, wherein the lock screen notification comprises an obscured display of the data on the lock screen.
EE 32. The method of EE 25, the method further comprising:
determining that the computing device is in a sleep state;
receiving a wake indicator before displaying the lock screen notification; and
waking the computing device from the sleep state and displaying the lock screen notification in response to the lock screen notification triggering event.
EE 33. The method of EE 25, wherein the lock screen notification removal event is one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.
EE 34. The method of EE 25, further comprising unlocking the screen and displaying a diabetes application in response to a selection of the lock screen notification.
EE 35. The method of EE 25, further comprising:
removing the diabetic lock screen notification in response to receipt of the user input.
EE 36. The method of EE 25, further comprising:
detecting, based on the received data, a lock screen notification removal event associated with the diabetic condition; and
in response to detecting the lock screen notification removal event, removing the diabetic lock screen notification from the lock screen.
EE 37. The method of EE 36, wherein in response to detecting the lock screen notification triggering event, the lock screen notification dynamically displays until identifying the received data that causes the removal of the lock screen notification.
EE 38. The method of EE 37, wherein the received data that causes the removal of the lock screen notification comprises an indication of the blood glucose level of the user coming within a predefined range associated with a normal glycemic state.
EE 39. An apparatus comprising:
a processor configured to perform the method as recited in any of EEs 1-13.
EE 40. An apparatus comprising:
a processor configured to perform the method as recited in any of EEs 14-24.
EE 41. An apparatus comprising:
a processor configured to perform the method as recited in any of EEs 25-38.
EE 42. A computer-readable medium having computer-executable instructions stored thereon that, when executed by a processor, cause the processor to perform the method as recited in any of EEs 1-13.
EE 43. A computer-readable medium having computer-executable instructions stored thereon that, when executed by a processor, cause the processor to perform the method as recited in any of EEs 14-24.
EE 44. A computer-readable medium having computer-executable instructions stored thereon that, when executed by a processor, cause the processor to perform the method as recited in any of EEs 25-38.

What is claimed is:

1. A method comprising:
receiving data related to a diabetic condition of a person;
detecting, based on the received data, an overlay notification triggering event associated with the diabetic condition;
in response to detecting the overlay notification triggering event, dynamically displaying a diabetic overlay notification on a graphical user interface of the computing device, wherein the diabetic overlay notification is associated with a diabetes application, wherein the overlay notification is displayed for a period of time or until an overlay notification removal event associated with the diabetic condition is detected, wherein the period of time is configured to be a predefined period of time from the overlay notification triggering event associated with the diabetic condition;
detecting, based on the received data related to the diabetic condition, the overlay notification removal event; and
in response to detecting the overlay notification removal event, removing the diabetic overlay notification from the graphical user interface of the computing device.

2. The method of claim 1, wherein the received data is data from a continuous glucose monitor.

3. The method of claim 2, wherein the received data is related to a chronic condition of the user for being monitored or device malfunctions.

4. The method of claim 2, wherein the overlay notification triggering event is detected when the received data from the continuous glucose monitor indicates a glucose level above an upper threshold or below a lower threshold, wherein the overlay notification removal event is detected when the received data from the continuous glucose monitor indicates a glucose level within the predefined range.

5. The method of claim 1, wherein the overlay notification triggering event is manually or automatically configured.

6. The method of claim 1, wherein the overlay notification triggering event is configured to be manually turned on or turned off.

7. The method of claim 1, wherein the overlay notification triggering event is detected based on one of an indication of an extreme diabetic state, an indication of an abnormal increase or decrease of blood glucose level within a predefined period of time, an indication of a performance of exercise by the person, an indication of mealtime, or an indication that the person received a predefined amount of short-acting insulin within a predefined period of time.

8. The method of claim 1, wherein the method further comprises:
determining that the computing device is in a sleep state;
receiving a wake indicator before displaying the overlay notification; and
waking the computing device from the sleep state and displaying the overlay notification in response to the overlay notification triggering event.

9. The method of claim 1, wherein the overlay notification removal event is manually or automatically configured.

10. The method of claim 9, wherein the overlay notification removal event is configured to be manually turned on or turned off.

11. The method of claim 1, wherein the overlay notification removal event is detected based on one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.

12. The method of claim 1, wherein the graphical user interface is one of a lock screen, a home screen, or a call screen displayed on the computing device.

13. The method of claim 12, wherein the graphical user interface is the lock screen, the method further comprising:
unlocking the screen and displaying a diabetes application in response to a selection of the overlay notification.

14.-24. (canceled)

25. A method comprising:
receiving data related to a diabetic condition of a person;
detecting, based on the received data, a lock screen notification triggering event associated with the diabetic condition;
in response to detecting the lock screen notification triggering event, providing a notification of data related to the diabetic condition or the person to be displayed on a lock screen of a computing device via a diabetic lock screen notification;
preventing display of the data related to the diabetic condition or the person until an input is received from a user to enable the display of the data;
receiving, via the lock screen, the input from the user to enable the display of the data related to the diabetic condition or the person on the lock screen; and
in response to the receipt of the input from the user, dynamically displaying the lock screen notification on the lock screen of the computing device.

26. The method of claim 25, wherein the received data is data from a continuous glucose monitor.

27. The method of claim 26, wherein the received data from the chronic condition than needs to be monitored or device malfunctions.

28. The method of claim 25, wherein the lock screen notification triggering event is manually configured.

29. The method of claim 25, wherein the lock screen notification triggering event is configured to be manually turned on or turned off.

30. The method of claim 25, wherein the lock screen notification triggering event is configured to be one of an extreme diabetic state, an abnormal increase or decrease of blood glucose level within a predefined period of time, an exercise, a meal, a snack or an indication that the person received short-acting insulin within a predefined period of time.

31. The method of claim 25, wherein the lock screen notification comprises an obscured display of the data on the lock screen.

32. The method of claim 25, the method further comprising:
determining that the computing device is in a sleep state;
receiving a wake indicator before displaying the lock screen notification; and
waking the computing device from the sleep state and displaying the lock screen notification in response to the lock screen notification triggering event.

33. The method of claim 25, wherein the lock screen notification removal event is one of an indication of a normal diabetic state, an indication of the blood glucose level stabilizing over a predefined period of time, an indication that the person is finished exercising, an indication that the person is finished having a meal, or an indication that the person's blood glucose level returned to within a predefined range.

34. The method of claim 25, further comprising unlocking the screen and displaying a diabetes application in response to a selection of the lock screen notification.

35. The method of claim 25, further comprising:
removing the diabetic lock screen notification in response to receipt of the user input.

36. The method of claim 25, further comprising:
detecting, based on the received data, a lock screen notification removal event associated with the diabetic condition; and
in response to detecting the lock screen notification removal event, removing the diabetic lock screen notification from the lock screen.

37. The method of claim 36, wherein in response to detecting the lock screen notification triggering event, the lock screen notification dynamically displays until identifying the received data that causes the removal of the lock screen notification.

38. The method of claim 37, wherein the received data that causes the removal of the lock screen notification comprises an indication of the blood glucose level of the user coming within a predefined range associated with a normal glycemic state.

39.-44. (canceled)
